SEQUENCE LISTING (10 sequences)

SEQ ID NO: 1 - DNA, 1826 bp, Human
aggactgtatgctgttcttaaggactctctgcttcctggacaagctcaagctaaggactacatctcccagcaggctgtgctctgacagctcttggatttaaataggattctgggctctgctcagagtcaggctgctgctcagcacccaggacggagaggagcagagaagcagcagaagcagccaagagctggagccagaccaggaacctgagccagagctggggttgaagctggagcagcagcaaaagcaacagcagctacagaagttggaacgatgctggtcaccttgggactgctcacctccttcttctcgttcctgtatatggtagctccatccatcaggaagttctttgctggtggagtgtgtagaacaaatgtgcagcttcctggcaaggtagtggtgatcactggcgccaacacgggcattggcaaggagacggccagagagctcgctagccgaggagcccgagtctatattgcctgcagagatgtactgaagggggagtctgctgccagtgaaatccgagtggatacaaagaactcccaggtgctggtgcggaaattggacctatccgacaccaaatctatccgagcctttgctgagggctttctggcagaggaaaagcagctccatattctgatcaacaatgcgggagtaatgatgtgtccatattccaagacagctgatggctttgaaacccacctgggagtcaaccacctgggccacttcctcctcacctacctgctcctggagcggctaaaggtgtctgcccctgcacgggtggttaatgtgtcctcggtggctcaccacattggcaagattcccttccacgacctccagagcgagaagcgctacagcaggggttttgcctattgccacagcaagctggccaatgtgctttttactcgtgagctggccaagaggctccaaggcaccggggtcaccacctacgcagtgcacccaggcgtcgtccgctctgagctggtccggcactcctccctgctctgcctgctctggcggctcttctccccctttgtcaagacggcacgggagggggcgcagaccagcctgcactgcgccctggctgagggcctggagcccctgagtggcaagtacttcagtgactgcaagaggacctgggtgtctccaagggcccgaaataacaaaacagctgagcgcctatggaatgtcagctgtgagcttctaggaatccggtgggagtagctggtggaagagctgcagctttatcaggcccaatccatgccataatgaacagggaccaaggagaaggccaaccctaaaggattgtcctcttggccagctggtgctgcgaatcctgcctgctctgatcctcttgacccttctgggaatgtttgcacacctgacactcttgtgagactggcttatggcatgagttgtggacacctatagagtgttcttctctaagacctggaaagtcagcaaccctctgggggcagcaggactgggcagatcccaggctgggcatgggggtggcagaagagcccgagaaattgggtcagttccctcatcagcaccagaggctcagctgaggcaagaagagcaccatcactgcctatttctaggggctatacactccaactcttggttgatctctttctttttaaaaatatttgccaccaccctggagtctagaccaacacacaaagatcctggctaaccctggcctatttagattccttcctctcacctggaccttcccatttcaatcatgcagatggtttctttttgtaaagagttccgtttgcctttcaatttttagagaaaataaagactgcattcatct

SEQ ID NO: 2 - protein, 316 aa, Human
MLVTLGLLTSFFSFLYMVAPSIRKFFAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFSDCKRTWVSPRARNNKTAERLWNVSCELLGIRWE

SEQ ID NO: 3 - DNA, 2538 bp, Human
gctggagcatcccgctctggtgccgctgcagccggcagagatggttgagctcatgttcccgctgttgctcctccttctgcccttccttctgtatatggctgcgccccaaatcaggaaaatgctgtccagtggggtgtgtacatcaactgttcagcttcctgggaaagtagttgtggtcacaggagctaatacaggtatcgggaaggagacagccaaagagctggctcagagaggagctcgagtatatttagcttgccgggatgtggaaaagggggaattggtggccaaagagatccagaccacgacagggaaccagcaggtgttggtgcggaaactggacctgtctgatactaagtctattcgagcttttgctaagggcttcttagctgaggaaaagcacctccacgttttgatcaacaatgcaggagtgatgatgtgtccgtactcgaagacagcagatggctttgagatgcacataggagtcaaccacttgggtcacttcctcctaacccatctgctgctagagaaactaaaggaatcagccccatcaaggatagtaaatgtgtcttccctcgcacatcacctgggaaggatccacttccataacctgcagggcgagaaattctacaatgcaggcctggcctactgtcacagcaagctagccaacatcctcttcacccaggaactggcccggagactaaaaggctctggcgttacgacgtattctgtacaccctggcacagtccaatctgaactggttcggcactcatctttcatgagatggatgtggtggcttttctcctttttcatcaagactcctcagcagggagcccagaccagcctgcactgtgccttaacagaaggtcttgagattctaagtgggaatcatttcagtgactgtcatgtggcatgggtgtctgtccaagctcgtaatgagactatagcaaggcggctgtgggacgtcagttgtgacctgctgggcctcccaatagactaacaggcagtgccagttggacccaagagaagactgcagcagactacacagtacttcttgtcaaaatgattctccttcaaggttttcaaaacctttagcacaaagagagcaaaaccttccagccttgcctgcttggtgtccagttaaaactcagtgtactgccagattcgtctaaatgtctgtcatgtccagatttactttgcttctgttactgccagagttactagagatatcataataggataagaagaccctcatatgacctgcacagctcattttccttctgaaagaaactactacctaggagaatctaagctatagcagggatgatttatgcaaatttgaactagcttctttgttcacaattcagttcctcccaaccaaccagtcttcacttcaagagggccacactgcaacctcagcttaacatgaataacaaagactggctcaggagcagggcttgcccaggcatggtggatcaccggaggtcagtagttcaagaccagcctggccaacatggtgaaaccccacctctactaaaaattgtgtatatctttgtgtgtcttcctgtttatgtgtgccaagggagtattttcacaaagttcaaaacagccacaataatcagagatggagcaaaccagtgccatccagtctttatgcaaatgaaatgctgcaaagggaagcagattctgtatatgttggtaactacccaccaagagcacatgggtagcagggaagaagtaaaaaaagagaaggagaatactggaagataatgcacaaaatgaagggactagttaaggattaactagccctttaaggattaactagttaaggattaatagcaaaagatattaaatatgctaacatagctatggaggaattgagggcaagcacccaggactgatgaggtcttaacaaaaaccagtgtggcaaaaaaaaaaaaaaaaaaaaaaaaaaaaaatcctaaaaacaaacaaacaaaaaaaacaattcttcattcagaaaaattatcttagggactgatattggtaattatggtcaatttaataatattttggggcatttccttacattgtcttgacaagattaaaatgtctgtgccaaaattttgtattttatttggagacttcttatcaaaagtaatgctgccaaaggaagtctaaggaattagtagtgttcccatcacttgtttggagtgtgctattctaaaagattttgatttcctggaatgacaattatattttaactttggtgggggaaagagttataggaccacagtcttcacttctgatacttgtaaattaatcttttattgcacttgttttgaccattaagctatatgtttagaaatggtcattttacggaaaaattagaaaaattctgataatagtgcagaataaatgaattaatgttttacttaatttatattgaactgtcaatgacaaataaaaattctttttgattattttttgttttcatttaccagaataaaaactaagaattaaaagtttgattacagtcaaaaaaaaaaaaaaaaaaaaaaa

SEQ ID NO: 4 - protein, 318 aa, Human
MVELMFPLLLLLLPFLLYMAAPQIRKMLSSGVCTSTVQLPGKVVVVTGANTGIGKETAKELAQRGARVYLACRDVEKGELVAKEIQTTTGNQQVLVRKLDLSDTKSIRAFAKGFLAEEKHLHVLINNAGVMMCPYSKTADGFEMHIGVNHLGHFLLTHLLLEKLKESAPSRIVNVSSLAHHLGRIHFHNLQGEKFYNAGLAYCHSKLANILFTQELARRLKGSGVTTYSVHPGTVQSELVRHSSFMRWMWWLFSFFIKTPQQGAQTSLHCALTEGLEILSGNHFSDCHVAWVSVQARNETIARRLWDVSCDLLGLPID

SEQ ID NO: 5 - DNA, 17 bp, Artificial Sequence
  Description of Artificial Sequence forward primer
caagggcccggaacaag

SEQ ID NO: 6 - DNA, 21 bp, Artificial Sequence
  Description of Artificial Sequence reverse primer
tggattcctagaagctcgcag

SEQ ID NO: 7 - DNA, 26 bp, Artificial Sequence
  Description of Artificial Sequence taqman probe
aacagctgagcgattgtggaacgtca

SEQ ID NO: 8 - DNA, 24 bp, Artificial Sequence
  Description of Artificial Sequence forward primer
aatccaggactacatttcccagag

SEQ ID NO: 9 - DNA, 20 bp, Artificial Sequence
  Description of Artificial Sequence reverse primer
cctagacacaccccggttca

SEQ ID NO: 10 - DNA, 22 bp, Artificial Sequence
  Description of Artificial Sequence taqman probe
ctttgctccctggcggttgcag

(19) United States
(12) Patent Application Publication
(10) Pub. No.: US 2005/0107317 A1
(43) Pub. Date: May 19, 2005

(54) Cg3842 homologous proteins involved in the regulation of energy homeostasis

(76) Inventors: Marcus Geese, Gottingen (DE); Martin Meise, Gottingen (DE); Bronner Gunter, Gottingen (DE)

Correspondence Address: Christopher B Linder;Thomas Kayden Horstemeyer & Risley, Suite 1750, 100 GalleriaParkway NW, Atlanta, GA, 30339-5948, US

(21) Appl. No.: 10/506,740

(22) PCT Filed: Mar. 7, 2003
(86) PCT No.: PCT/EP03/02363

(30) Foreign Application Priority Data
Mar. 7, 2002 (EP) 02 005 141.3

(51) Int. Cl. A61K 48/00; C12Q 1/68; A61K 38/44

(52) U.S. Cl. 514/44; 424/94.4; 435/6

(57) ABSTRACT

The present invention discloses CG3842 or SCAD homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of metabolic diseases and disorders.

This invention relates to the use of nucleic acid sequences encoding CG3842 or SCAD homologous proteins, and the polypeptides encoded thereby and to the use thereof or effector molecules of CG3842 or SCAD homologous nucleic acids or polypeptides in the diagnosis, study, prevention, and treatment of diseases and disorders related to body-weight regulation, for example, but not limited to, metabolic diseases such as obesity as well as related disorders such as metabolic syndrome eating disorder, cachexia, diabetes mellitus, hypertension, coronary heart disease, hypercholesterolemia, dyslipidemia, osteoarthritis, and/or gallstones.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still poorly understood human disease that becomes more and more relevant for western society. Obesity is defined as an excess of body fat, frequently resulting in a significant impairment of health. Cardiovascular risk factors like hypertension, high blood levels of triglycerides and fasting glucose as well as low blood levels of HDL cholesterol are often linked to obesity. This typical cluster of symptoms is commonly defined as “metabolic syndrome” (Reaven, 2002, Circulation 106(3): 286-8). Hyperlipidemia and elevation of free fatty acids correlate clearly with the metabolic syndrome, which is considered to be the linkage between several diseases, including obesity and insulin resistance. This often occurs in the same patients and is a major risk factor for development of Type 2 diabetes and cardiovascular disease. It was suggested that the control of lipid levels and glucose levels is required to treat Type 2 Diabetes, heart disease, and other occurances of metabolic syndrome (see, for example, Santomauro A. T. et al., (1999) Diabetes, 48(9):1836-1841, McCook, 2002, JAMA 288:2709-2716).

Human obesity is strongly influenced by environmental and genetic factors, whereby the environmental influence is often a hurdle for the identification of (human) obesity genes. Obesity is influenced by genetic, metabolic, biochemical, psychological, and behavioral factors. As such, it is a complex disorder that must be addressed on several fronts to achieve lasting positive clinical outcome.

Obesity is not to be considered as a single disorder but a heterogeneous group of conditions with (potential) multiple causes. Obesity is also characterized by elevated fasting plasma insulin and an exaggerated insulin response to oral glucose intake (Koltermann, 1980, J. Clin. Invest 65:1272-1284). A clear involvement of obesity in type 2 diabetes mellitus can be confirmed (Kopelman, 2000, Nature 404:635-643).

Insulin amongst other hormones plays a key role in the regulation of the fuel metabolism. High blood glucose levels stimulate the secretion of insulin by pancreatic beta-cells. Insulin leads to the storage of glycogen and triglycerides and to the synthesis of proteins. The entry of glucose into muscles and adipose cells is stimulated by insulin. In patients who suffer from diabetes mellitus either the amount of insulin produced by the pancreatic islet cells is to low (Diabetes Type 1 or insulin dependent diabetes mellitus, IDDM) or liver and muscle cells loose their ability to respond to normal blood insulin levels (insulin resistance). In the next stage pancreatic cells become unable to produce sufficient amounts of insulin (Diabetes Type II or non insulin dependent diabetes mellitus NIDDM).

Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass (and thus metabolic syndrome) regulations are not known.

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses a specific gene involved in the regulation of body-weight, energy homeostasis, metabolism, and obesity, and thus in disorders related thereto such as metabolic syndrome, eating disorder, cachexia, diabetes mellitus, hypertension, coronary heart disease, hypercholesterolemia, dyslipidemia, osteoarthritis, and gallstones. The present invention describes the human homologs of the Drosophila CG3842 gene as being involved in those conditions mentioned above.

The acyl-CoA dehydrogenase (Acad or ACAD) gene family of enzymes includes very-long-chain (VLCAD), medium-chain (MCAD), and short-chain (SCAD) acyl-CoA dehydrogenases. The short-chain dehydrogenases/reductases family (SDR) constitute a large and diverse family of enzymes of ancient origin. Several of its members play an important role in human physiology and disease, especially in the metabolism of steroid substrates (e.g., prostaglandins, estrogens, retinoids, androgens, and corticosteroids). Their involvement in common human disorders such as endocrine-related cancer, osteoporosis, and Alzheimer disease makes them important candidates for drug targets.

The Drosophila gene of this invention (GadFly Accession Number CG3842) has one aminoterminal transmembrane domain and to a large extend exhibits a predicted secondary structure motif characteristic of short-chain alcohol dehydrogenases (adh-short motif; e.g., from amino acid 73 to amino acid 328 in the protein of 406 amino acids length). Three human homologous proteins were identified in this invention. These proteins are the unnamed protein XP_{—}085058 (BAB70811, also referred to as DG21-1 herein) and CGI-82 (also referred to as prostate short-chain dehydrogenase reductase 1, PSDR 1, or DG21-2 herein), and also PAN2.

The human PSDR1 (CGI-82) gene was identified by comparative genomics (Lai et al., 2000, Genome Res 10(5):703-713). PSDR1 is highly expressed in the prostate gland and a function in the androgen receptor-regulated gene network of the human prostate was suggested. Genes regulated by androgenic hormones are of critical importance for the normal physiological function of the human prostate gland, and they contribute to the development and progression of prostate carcinoma (Lin et al., 2001, Cancer Res 61(4):1611-1618).

Human BAB70811 (unnamed protein XP_{—}085058) cDNA was isolated as part of a sequencing project from human brain (cerebrellum) tissue. No functional data were available at the time the invention was made.

The human PAN2 protein has been submitted to the NCBI Genbank recently (GenBank Accession Number NP_{—}065965; submitted Feb. 10, 2002 by Brereton et al.). PAN2 has been described as member of the SCAD superfamily.

So far, it has not been described that CG3842 encoded protein and closely related proteins, particularly human proteins unnamed protein XP_{—}085058, CGI-82, and PAN2, are involved in the regulation of energy homeostasis and body-weight regulation and related disorders, and thus, no functions in metabolic diseases and other diseases as listed above have been discussed.

In this invention we demonstrate that the correct gene dose of CG3842 is essential for maintenance of energy homeostasis. A genetic screen was used to identify that mutation of a CG3842 homologous gene causes obesity, reflected by a significant increase of triglyceride content, the major energy storage substance.

Polynucleotides encoding a protein with homologies to CG3842 are suitable to investigate diseases and disorders as described above. Further new compositions useful in diagnosis, treatment, and prognosis of diseases and disorders as described above are provided.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure.

The present invention discloses that CG3842 homologous proteins are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these polynucleotides, polypeptides and effectors thereof, e.g. antibodies, aptamers, anti-sense molecules, ribozymes or other receptors recognizing a nucleic acid molecule or polypeptide homologous to CG3842 in the diagnosis, study, prevention, and treatment of diseases and disorders, for example, but not limited to, metabolic diseases such as obesity as well as related disorders such as metabolic syndrome, eating disorder, cachexia, diabetes mellitus, hypertension, coronary heart disease, hypercholesterolemia, dyslipidemia, osteoarthritis and/or gallstones.

The term “polynucleotide comprising the nucleotide sequence as shown in GenBank Accession number” relates to the expressible gene of the nucleotide sequences deposited under the corresponding GenBank Accession number. The term “GenBank Accession Number” relates to NCBI GenBank database entries (Ref.: Benson et al., (2000) Nucleic Acids Res. 28: 15-18).

CG3842 homologous proteins and nucleic acid molecules coding therefore are obtainable from insect or vertebrate species, e.g. mammals or birds. Particularly preferred are human homologous nucleic acids, particularly nucleic acids encoding a human unnamed protein, a human CGI-82 protein, or PAN2 protein.

The invention particularly relates to a nucleic acid molecule encoding a polypeptide contributing to regulating the energy homeostasis and the metabolism of triglycerides, wherein said nucleic acid molecule comprises
(a) the nucleotide sequence of or a nucleotide sequence encoding an unnamed protein (SEQ ID NO: 1; GenBank Accession Number XM_{—}085058), human CGI-82 (SEQ ID NO: 3; GenBank Accession Number NM_{—}016026), or PAN2 (GenBank Accession Number NM_{—}020905), or GadFly Accession Number CG3842 and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99,6% identical to the amino acid sequence of CG3842 homologous proteins,
(e) a sequence encoding a CG3842 homologous protein, preferably a human CG3842 homologous protein unnamed protein’ with SEQ ID NO: 2; GenBank Accession Number XP_{—}085058), CGI-82 protein (SEQ ID NO: 4; GenBank Accession Number NP 057110), or PAN2 (GenBank Accession Number NP_{—}065956), and/or a sequence complementary thereto,
(f) a sequence which differs from the nucleic acid molecule of (a) to (d) by mutation and wherein said mutation causes an alteration, deletion, duplication and/or premature stop in the encoded polypeptide or
(g) a partial sequence of any of the nucleotide sequences of (a) to (e) having a length of at least 15 bases, preferably at least 20 bases, more preferably at least 25 bases and most preferably at least 50 bases.

The invention is based on the finding that CG3842 homologous proteins, particularly proteins of the SCAD family as defined above (herein referred to as CG3842 or CG3842 homologous proteins), and the polynucleotides encoding these are involved in the regulation of triglyceride storage and therefore energy homeostasis. The invention describes the use of these compositions for the diagnosis, study, prevention, or treatment of diseases and disorders related thereto, including metabolic syndrome.

Accordingly; the present invention relates to genes with novel functions in body-weight regulation, energy homeostasis, metabolism, and obesity, fragments of said genes, polypeptides encoded by said genes or fragments thereof, and effectors e.g. antibodies, biologically active nucleic acids, such as antisense molecules, RNAi molecules or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease. In one embodiment, a forward genetic screen is performed in fly displaying a mutant phenotype due to misexpression of a known gene (see, Johnston Nat Rev Genet 3: 176-188 (2002); Rorth P., (1996) Proc Natl Acad Sci USA 93: 12418-12422).

Triglycerides are the most efficient storage for energy in cells, and obese people mainly show a significant increase in the content of triglycerides. In this invention, we have used a genetic screen to identify mutations that cause changes in the body weight which is reflected by a significant change of triglyceride levels. To isolate genes with a function in energy homeostasis, several thousand EP-lines were tested for their triglyceride content after a prolonged feeding period (see Examples for more detail). Lines with significantly changed triglyceride content were selected as positive candidates for further analysis. The change of triglyceride content due to the loss of a gene function suggests gene activities in energy homeostasis in a dose dependent manner that control the amount of energy stored as triglycerides.

A resource for screening was a proprietary Drosophila melanogaster stock collection of PX-lines. The P-vector of this collection has Gal4-UAS-binding sites fused to a basal promoter that can transcribe adjacent genomic Drosophila sequences upon binding of Gal4 to UAS-sites. This enables the PX-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

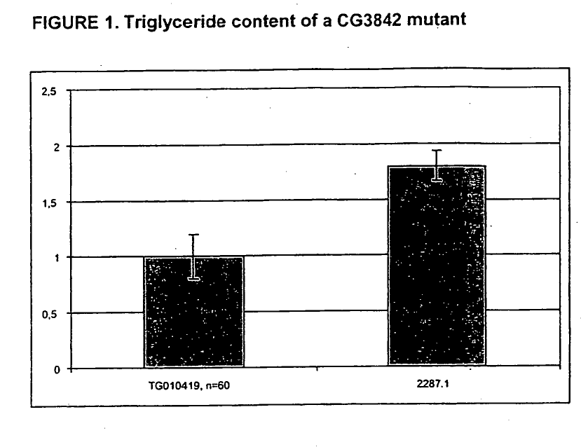

In one embodiment, flies homozygous for the integration of vectors for Drosophila line PX2287.1 were analyzed in an assay measuring the triglyceride contents of these flies, illustrated in more detail in the EXAMPLES section of the invention. The result of the triglyceride content analysis is shown in FIG. 1. The average triglyceride level of the fly collection in which the PX2287.1 line was found is shown as 100% in FIG. 1 (First column, “TG010419, n=60”). The average increase of triglyceride content of the homozygous viable Drosophila line PX2287.1 is 80% (see FIG. 1, second column, line “2287.1”). It was found in this invention that homozygous PX2287.1 flies have a significant higher triglyceride content than the control flies tested. The increase of triglyceride content due to the loss of a gene function suggests gene activities in energy homeostasis in a dose dependent manner that controls the amount of energy stored as triglycerides.

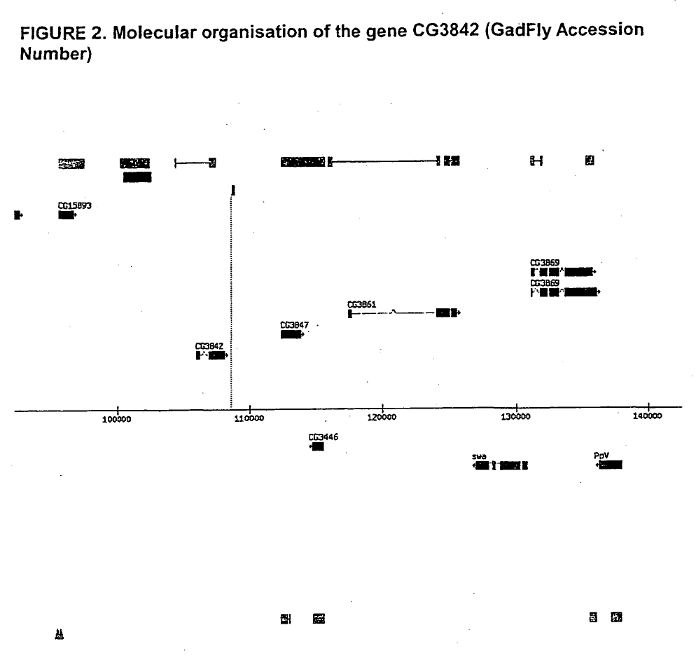

Nucleic acids encoding the Drosophila GadFly Accession Number CG3842 homologous proteins of the present invention were identified using an iPCR technique. Genomic DNA sequences were isolated that are localized adjacent to the EP vector (herein PX2287.1) integration. Using those isolated genomic sequences public databases like Berkeley Drosophila Genome Project (GadFly; see also FlyBase (1999) Nucleic Acids Research 27:85-88) or GenBank (NCBI) were screened thereby confirming the homozygous viable integration site of the PX2287.1 vector 542 base pairs downstream of the coding seqence of a gene, identified as Berkeley Drosophila Genome Project Accession Nr. CG3842 (FIG. 2). FIG. 2 shows the molecular organization of this gene locus.

Therefore, expression of the cDNA encoding GadFly Accession Number CG3842 could be effected by homozygous viable integration of vectors of line PX2287.1, leading to increase of the energy storage triglycerides.

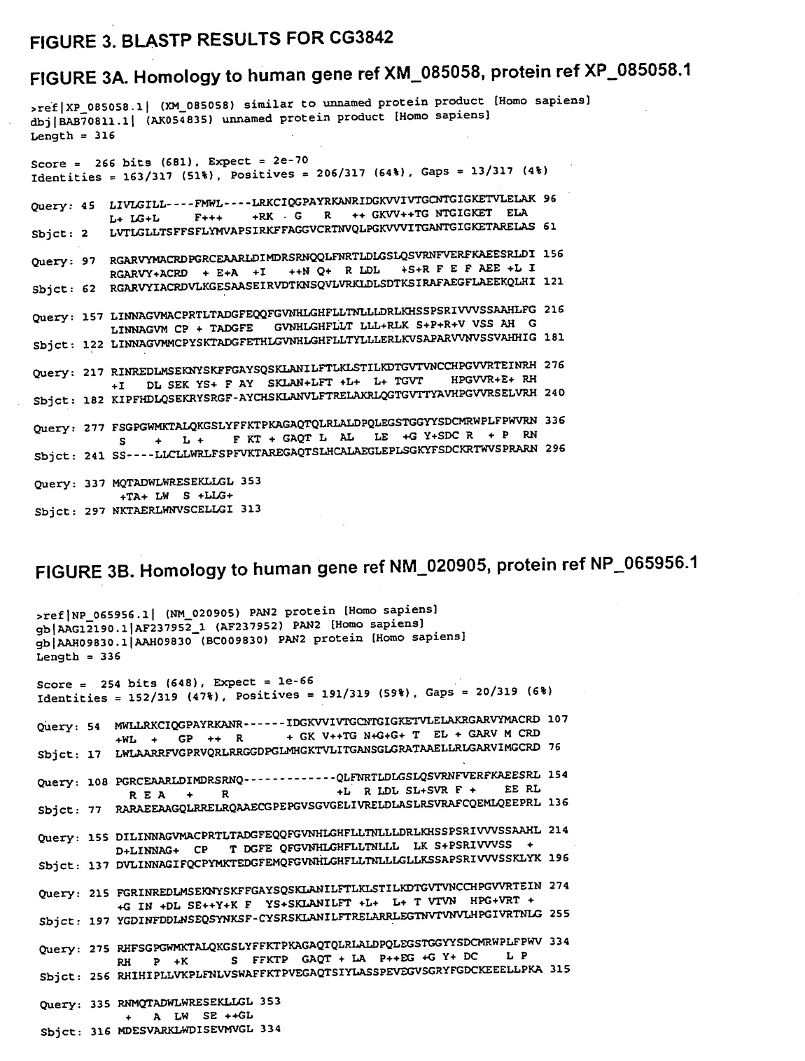

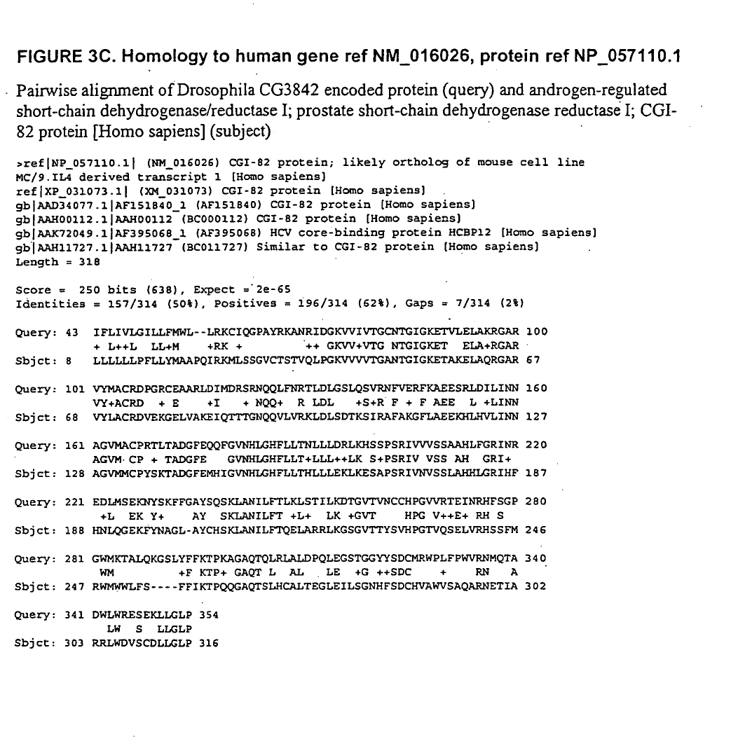

The Drosophila genes and proteins encoded thereby with functions in the regulation of triglyceride metabolism were further analysed in publicly available sequence databases (see EXAMPLES for more detail) and mammalian homologs were identified (see FIG. 3).

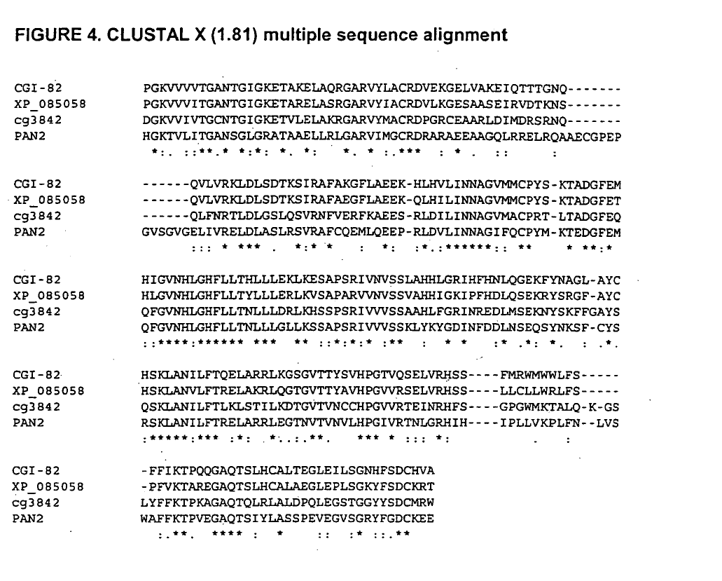

The present invention is further describing a polypeptide comprising the amino acid sequence of CG3842. A comparison (Clustal X (1.81) analysis) between the CG3842 proteins of different species (human and Drosophila) was conducted (see FIG. 4). Based upon homology, CG3842 protein of the invention and each homologous protein or peptide may share at least some activity. No functional data described the regulation of body weight control and related metabolic diseases such as obesity and diabetes are available in the prior art for the genes of the invention.

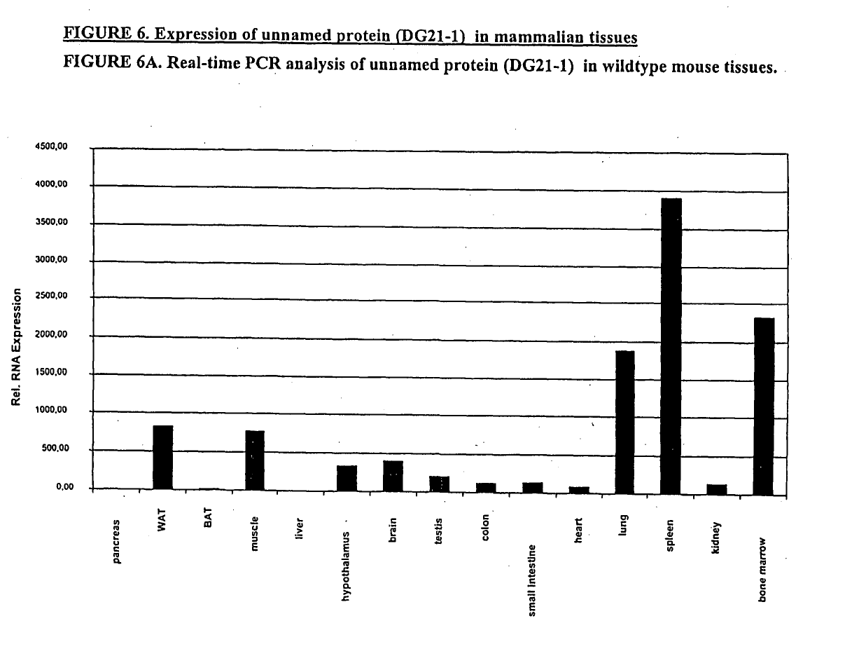

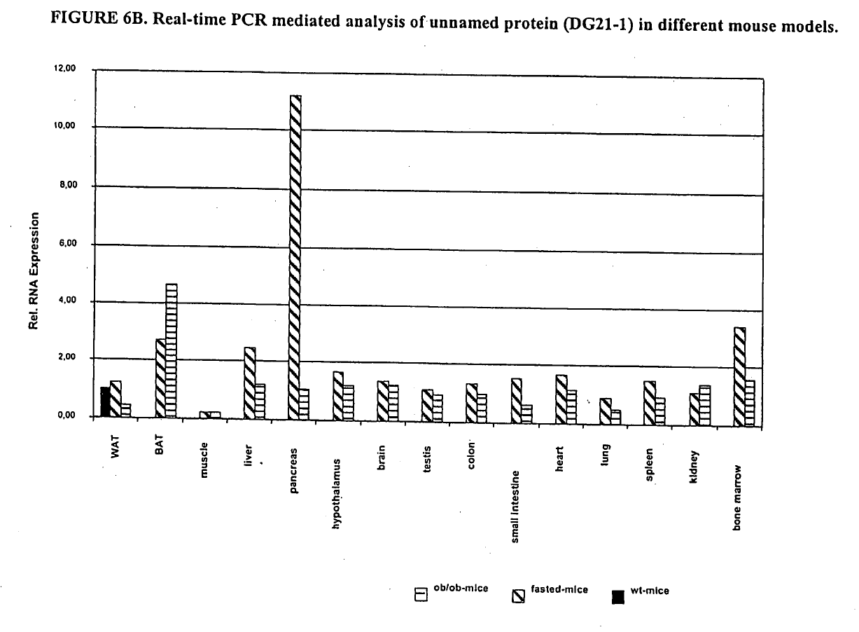

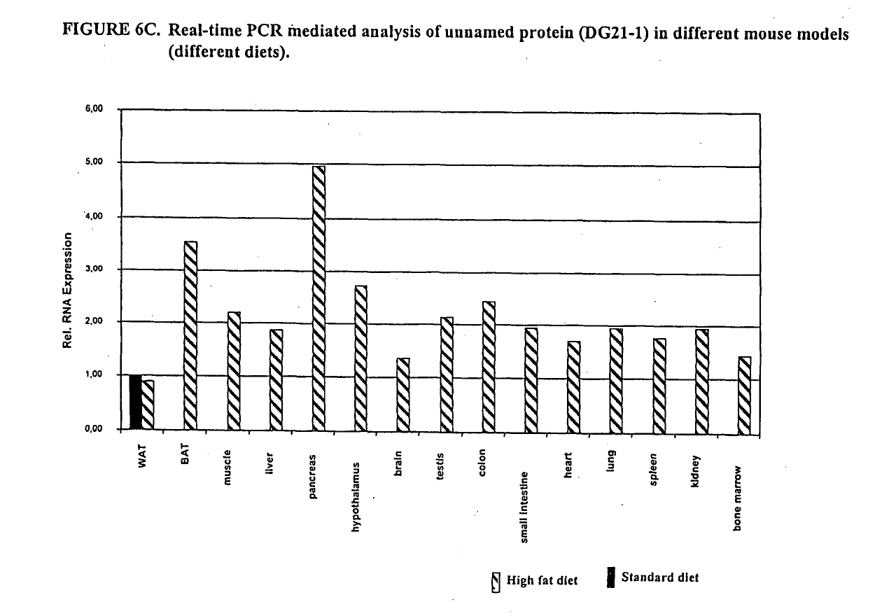

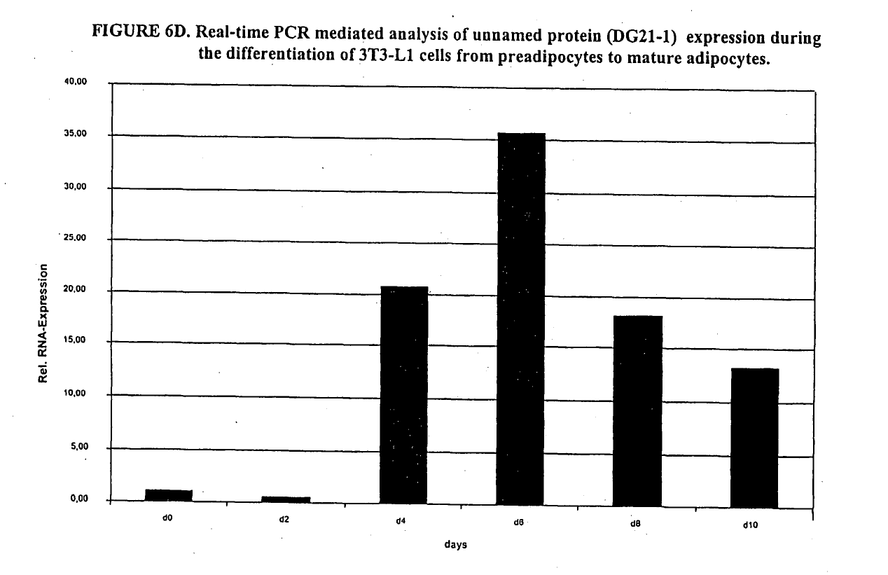

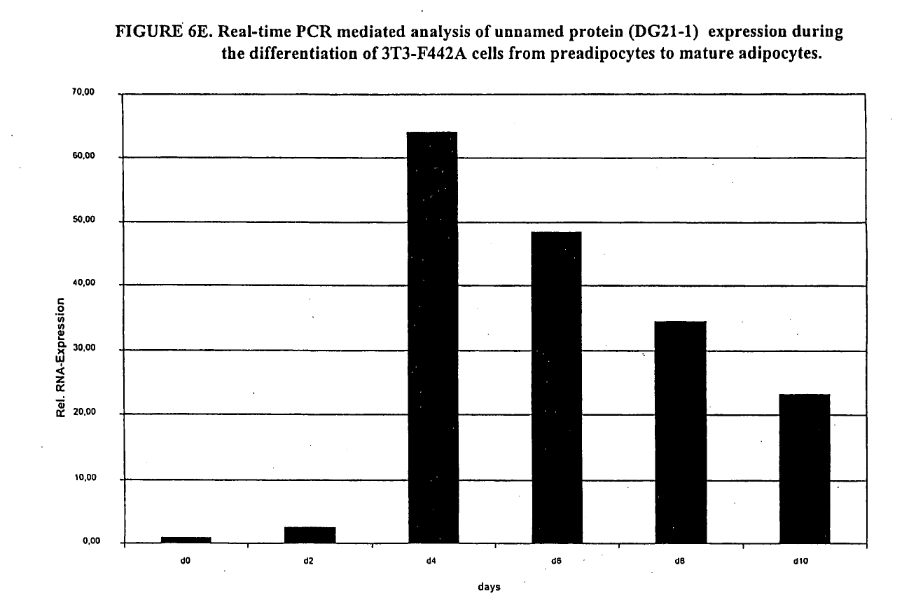

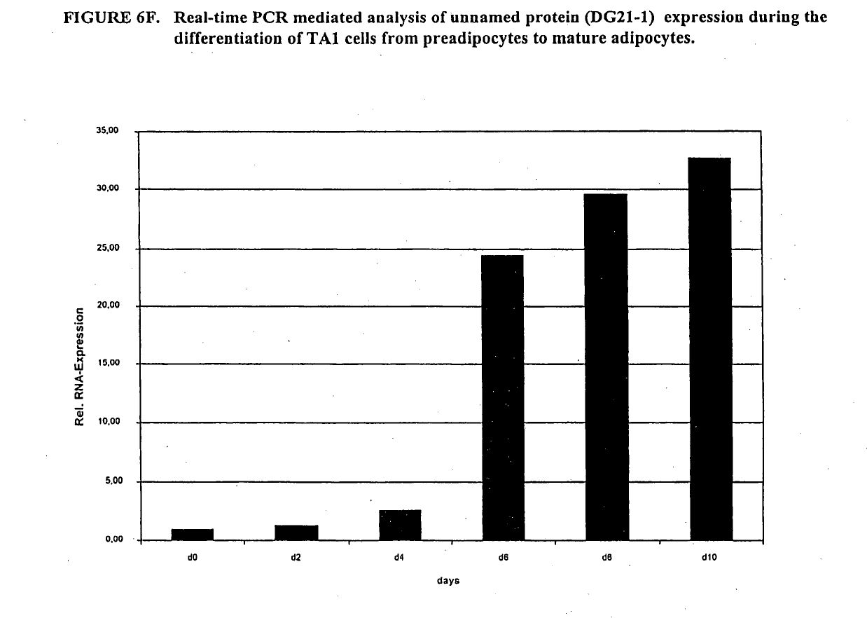

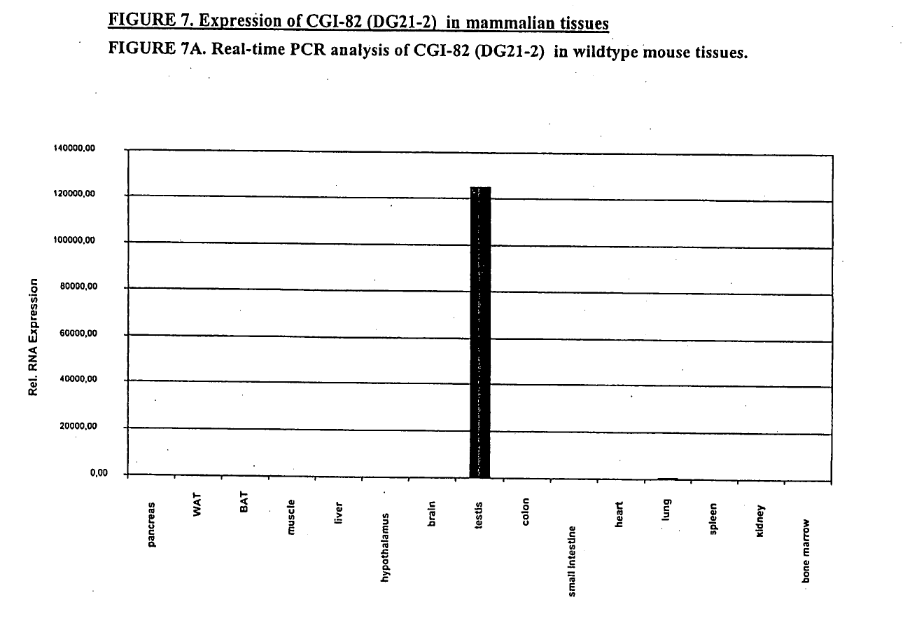

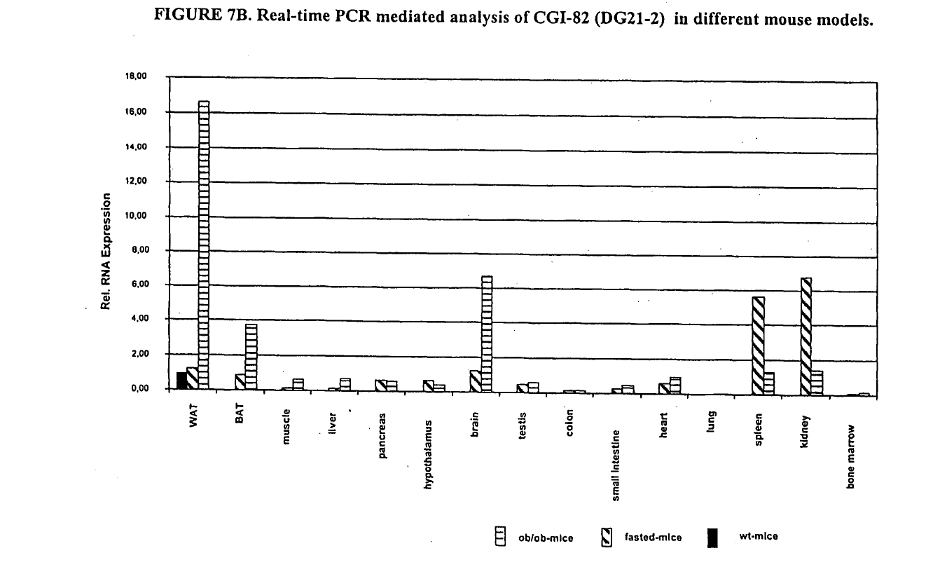

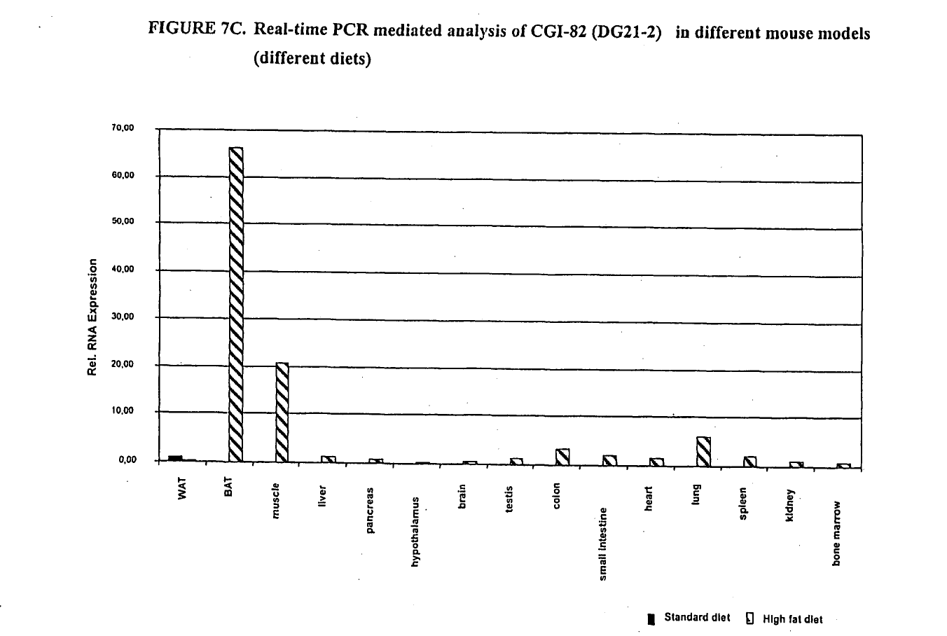

The function of the mammalian homologs in energy homeostasis was further validated in this invention by analyzing the expression of the transcripts in different tissues and by analyzing the role in adipocyte differentiation. Expression profiling studies (see EXAMPLES for more detail) confirm the particular relevance of the protein(s) of the invention as regulators of energy metabolism in mammals. For example, transcripts of unnamed protein (DG21-1) are more restricted to spleen, bone marrow and lung of mammals (FIG. 6A). CGI-82 (DG21-2) transcripts show highest expression in testis (FIG. 7A). In addition, the proteins of the invention are also clearly expressed in white adipose tissue (WAT); unnamed protein shows high levels of expression especially in WAT (see FIG. 6A). Thus an expression of the protein of the invention in adipose tissues is confirming a role in the regulation of energy homeostasis and thermogenesis.

Further, we show that the proteins of the invention are regulated by fasting and by genetically induced obesity. In this invention, we used mouse models of insulin resistance and/or diabetes, such as mice carrying gene knockouts in the leptin pathway (for example, ob (leptin) or db (leptin receptor) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569). We found, for example, that the expression of unnamed protein (DG21-1) is strongly upregulated especially in the pancreas of fasted mice (see FIG. 6B). The expression of CGI-82 (DG21-2) is strongly upregulated in spleen and kidney of fasted mice (FIG. 7B). In addition, a marked upregulation of DG21-1 and DG21-2 can be observed in the metabolically active tissue (for example, DG21-1 in brown adipose tissue (BAT) and DG21-2 in WAT) of genetically obese (ob/ob) as well as of fasted mice (see FIG. 6B, and FIG. 7B, respectively).

In addition, DG21-1 (unnamed protein) mRNA is upregulated in pancreas and BAT of mice with symptoms of diabetes, lipid accumulation, and high plasma lipid levels, if fed a high fat diet (HFD) (FIG. 6C). DG-21-2 (homolog to CGI-82=PSDR1) is strongly up regulated in white adipose tissue (WAT) of ob/ob mice compared to wt mice (FIG. 7B) and also in BAT and muscle of mice fed a HFD compared to mice fed a standard diet (FIG. 7C). These results provide evidence that the proteins of the invention are modulators of adipogenesis.

In addition, we show in this invention that the mRNA of unknown protein (DG21-1) is significantly up-regulated during adipocyte differentiation in vitro (see EXAMPLES for more detail, see FIG. 6D, FIG. 6E, FIG. 6F), suggesting a role as modulator of adipocyte lipid accumulation. Thus, we conclude that the protein of the invention (or variants thereof) have a function in the metabolism of mature mammalian adipocytes.

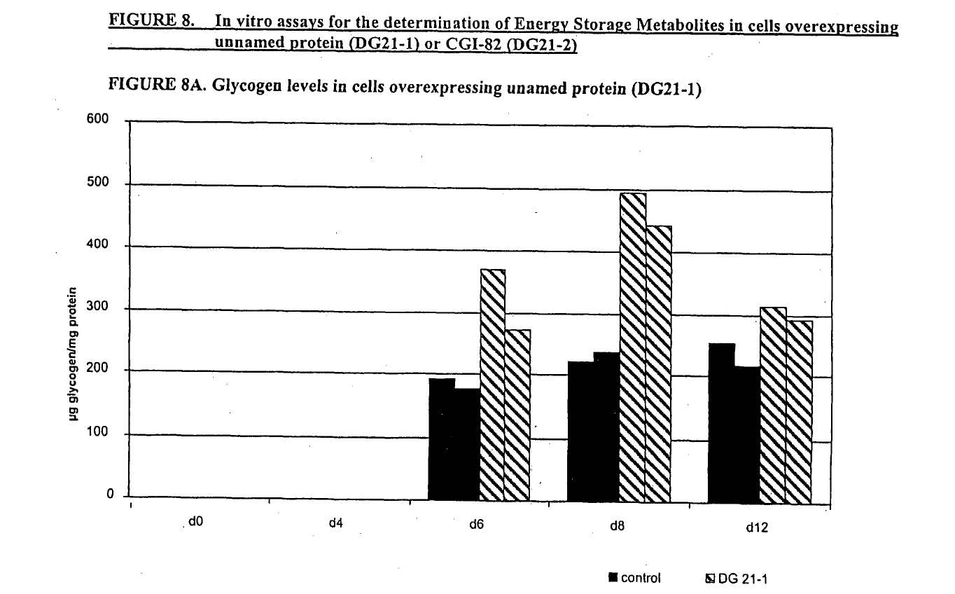

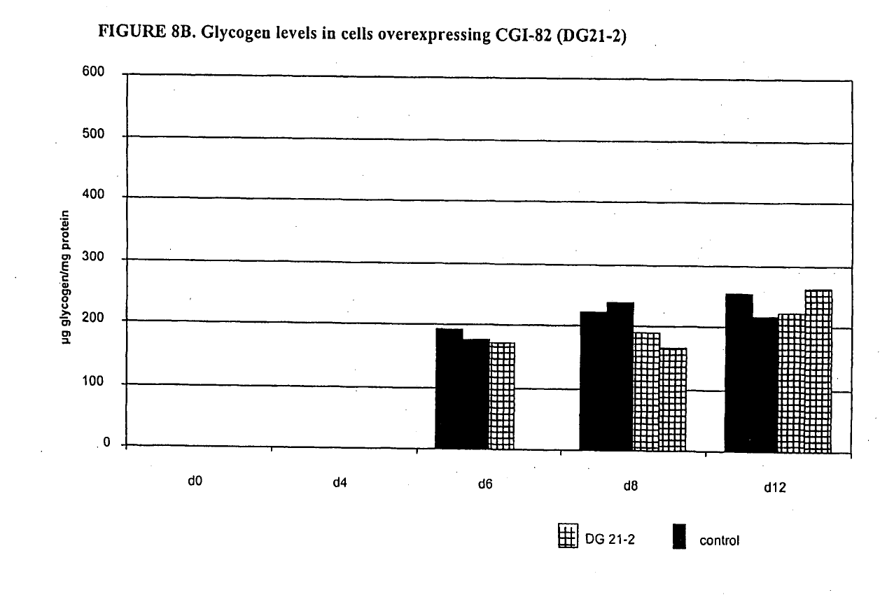

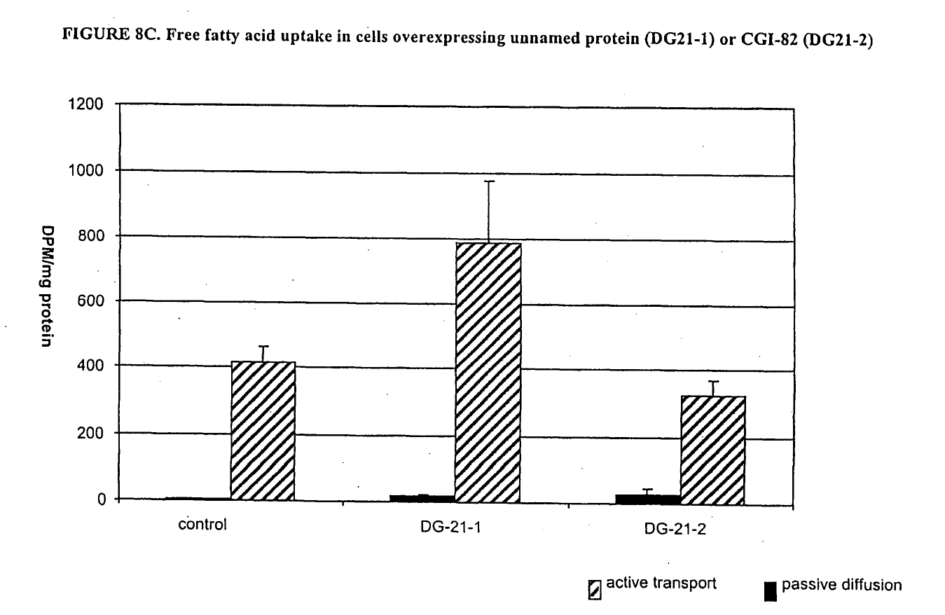

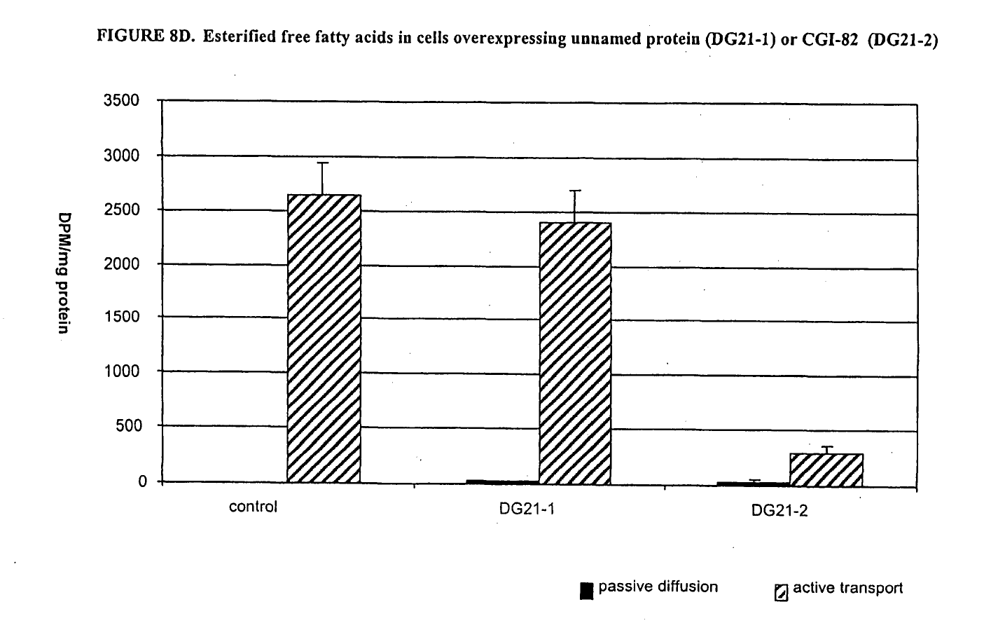

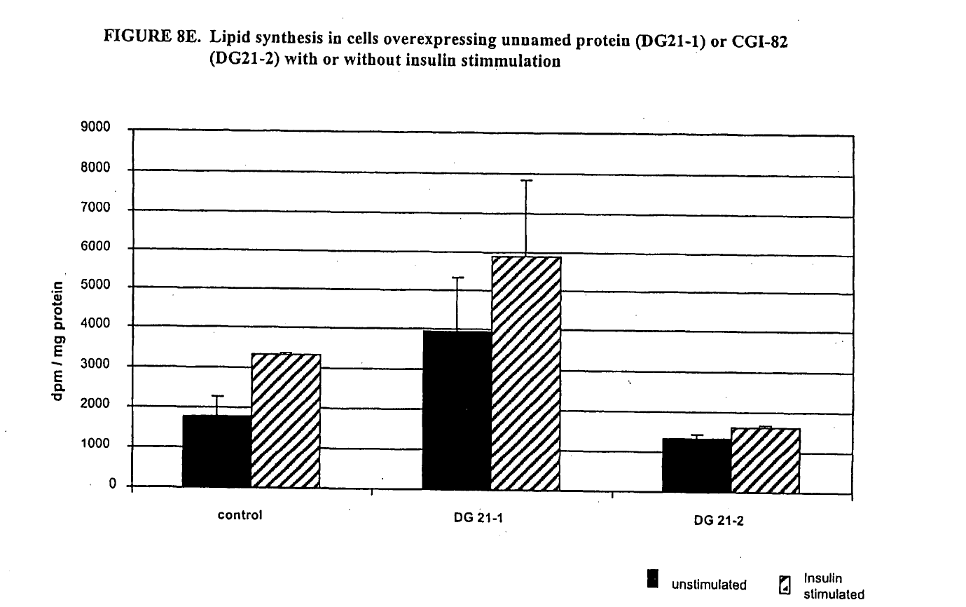

Glucose is taken up by the cells rapidly and stored in the form of glycogen primarily used for the metabolic demands of the cell. An increase in cellular glycogen levels as a consequence of overexpression of unnamed protein XP_{—}085058 (see FIG. 8A) could reflect an elevated glucose uptake, a higher glycogen synthesis rate or a decreased energy consumption and thus confirms a role of unnamed protein XP_{—}085058 in metabolic regulation.

In addition, we show in this invention that cells overexpressing unnamed protein XP_{—}085058 are more effective in synthesising lipids from exogenous glucose (see FIG. 8E). Consequently, levels of insulin stimulated lipid synthesis of cells overexpressing unnamed protein XP_{—}085058 are also increased. The increased level of lipid synthesis from exogenous glucose in cells overexpressing unnamed protein XP_{—}085058 demonstrate an activation of enzymes involved in lipid synthesis.

The profound increase in uptake of free fatty acids of cells overexpressing unnamed protein XP_{—}085058 (see FIG. 8C) and the decrease of the free fatty acid esterification in CGI-82 overexpressing cells (see FIG. 8D) could be due to a direct action of the proteins of the invention or alternatively due to a role in the regulation of free fatty acid uptake and esterification. The proteins of the invention may for example play a role in converting regulatory inactive retinoids or steroids in regulatory active hormone derivatives which then influence fatty acid metabolism directly or regulate the gene expression of fatty acid metabolic enzymes or transporters on the transcriptional level.

The invention also encompasses polynucleotides that encode CG3842 and homologous proteins. Accordingly, any nucleic acid sequence, which encodes the amino acid sequences of CG3842 homologous proteins, can be used to generate recombinant molecules that express CG3842 homologous proteins. It will be appreciated by those skilled in the art that as a result of the degeneracy of the genetic code, a multitude of nucleotide sequences encoding CG3842 homologous proteins, some bearing minimal homology to the nucleotide sequences of any known and naturally occurring gene, may be produced. Thus, the invention contemplates each and every possible variation of nucleotide sequence that could be made by selecting combinations based on possible codon choices.

Also encompassed by the invention are polynucleotide sequences that are capable of hybridizing to the claimed nucleotide sequences, and in particular, those of the polynucleotides comprising the nucleic acid sequence encoding a Drosophila protein (GadFly Accession Number CG3842) and homologous human proteins. Hybridization conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding CG3842, which are encompassed by the invention include deletions, insertions, or substitutions of different nucleotides resulting in a polynucleotide that encodes the same or a functionally equivalent CG3842 homologous proteins.

The encoded proteins may also contain deletions, insertions, or substitutions of amino acid residues, which produce a silent change and result in a functionally equivalent CG3842 homologous proteins. Deliberate amino acid substitutions may be made on the basis of similarity in polarity, charge, solubility, hydrophobicity, hydrophilicity, and/or the amphipathic nature of the residues as long as the biological activity of CG3842 homologous proteins is retained. Furthermore, the invention relates to peptide fragments of the proteins or derivatives thereof such as cyclic peptides, retro-inverso peptides or peptide mimetics having a length of at least 4, preferably at least 6 and up to 50 amino acids.

Also included within the scope of the present invention are alleles of the genes encoding CG3842 homologous proteins. As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides.

Each of these types of changes may occur alone, or in combination with the others, one or more times in a given sequence.

The nucleic acid sequences encoding CG3842 homologous proteins may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method, which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express a biologically active CG3842 homologous proteins, the nucleotide sequences encoding CG3842 homologous proteins or functional equivalents, optionally fused to heterologous sequences may be inserted into appropriate expression vectors, i.e., a vector, which contains the necessary elements for the transcription and translation of the inserted coding sequence. Methods, which are well known to those skilled in the art, may be used to construct expression vectors containing sequences encoding CG3842 homologous proteins and appropriate transcriptional and translational control elements. These methods include in vitro recombinant DNA techniques, synthetic techniques, and in vivo genetic recombination.

Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding CG3842 homologous proteins. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g. baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences encoding CG3842 homologous proteins can be detected by DNA-DNA or DNA-RNA hybridization and/or amplification using probes or portions or fragments of polynucleotides specific for CG3842 homologous proteins. Nucleic acid amplification based assays involve the use of oligonucleotides or oligomers based on the sequences encoding CG3842 homologous proteins to detect transformants containing DNA or RNA encoding CG3842 homologous proteins. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of CG3842 homologous proteins, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on CG3842 homologous proteins is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding CG3842 homologous proteins include oligo-labeling, nick translation, end-labeling or PCR amplification using a labeled nucleotide.

Alternatively, the sequences encoding CG3842 homologous proteins, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding CG3842 homologous proteins may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode CG3842 homologous proteins may be designed to contain signal sequences, which direct secretion of CG3842 homologous proteins through a prokaryotic or eukaryotic cell membrane. Other recombinant constructions may be used to join sequences encoding CG3842 homologous proteins to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and CG3842 homologous proteins may be used to facilitate purification. In addition to recombinant production, fragments of CG3842 homologous proteins may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesizer (Perkin Elmer). Various fragments of CG3842 homologous proteins may be chemically synthesized separately and combined using chemical methods to produce the full length molecule.

Diagnostics and Therapeutics

The data disclosed in this invention show that the nucleic acids and proteins of the invention and effector molecules thereof are useful in diagnostic and therapeutic applications implicated, for example but not limited to, in metabolic disorders such as obesity as well as related disorders such as eating disorder, cachexia, diabetes mellitus, hypertension, coronary heart disease, hypercholesterolemia, dyslipidemia, osteoarthritis and/or gallstones. Hence, diagnostic and therapeutic uses for the CG3842 homologous proteins nucleic acids and proteins of the invention are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in diagnostic and therapeutic applications implicated in various applications as described below. For example, but not limited to, cDNAs encoding the CG3842 proteins of the invention and particularly their human homologues may be useful in gene therapy, and the CG3842 proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The novel nucleic acid encoding the CG3842 homologous proteins of the invention, or fragments thereof, may further be useful in diagnostic applications, wherein the presence or amount of the nucleic acids or the proteins are to be assessed. These materials are further useful in the generation of antibodies that bind immunospecifically to the novel substances of the invention for use in therapeutic or diagnostic methods.

For example, in one aspect, antibodies that are specific for CG3842 homologous proteins may be used directly as an antagonist, or indirectly as a targeting or delivery mechanism for bringing a pharmaceutical agent to cells or tissue which express CG3842 homologous proteins. The antibodies may be generated using methods that are well known in the art. Such antibodies may include, but are not limited to, polyclonal, monoclonal, chimerical, single chain, Fab fragments, and fragments produced by a Fab expression library. Neutralizing antibodies (i.e., those which inhibit dimer formation) are especially preferred for therapeutic use.

For the production of antibodies, various hosts including goats, rabbits, rats, mice, humans, and others, may be immunized by injection with CG3842 homologous proteins any fragment or oligopeptide thereof which has immunogenic properties. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable. It is preferred that the peptides, fragments, or oligopeptides used to induce antibodies to CG3842 homologous proteins have an amino acid sequence consisting of at least five amino acids, and more preferably at least 10 amino acids. It is preferable that they are identical to a portion of the amino acid sequence of the natural protein, and they may contain the entire amino acid sequence of a small, naturally occurring molecule. Short stretches of CG3842 homologous proteins amino acids may be fused with those of another protein such as keyhole limpet hemocyanin and antibody produced against the chimeric molecule.

Monoclonal antibodies to CG3842 homologous proteins may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce CG3842 homologous proteins-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments, which contain specific binding sites for CG3842 homologous proteins, may also be generated. For example, such fragments include, but are not limited to, the F(ab′)₂ fragments which can be produced by pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between CG3842 homologous proteins and its specific antibody. A two-site, monoclonal-based immunoassay utilizing monoclonal antibodies reactive to two non-interfering CG3842 homologous proteins epitopes is preferred, but a competitive binding assay may also be employed (Maddox, supra).

In another embodiment of the invention, the polynucleotides encoding CG3842 homologous proteins, or any fragment thereof, or nucleic acid effector molecules such as antisense molecules, aptamers, RNAi molecules or ribozymes may be used for therapeutic purposes. In one aspect, aptamers, i.e. nucleic acid molecules, which are capable of binding to a CG3842 protein and modulating its activity, may be generated by a screening and selection procedure involving the use of combinatorial nucleic acid libraries.

In a further aspect, antisense to the polynucleotide encoding CG3842 homologous proteins may be used in situations in which it would be desirable to block the transcription of the mRNA. In particular, cells may be transformed with sequences complementary to polynucleotides encoding CG3842 homologous proteins. Thus, antisense molecules may be used to modulate CG3842 homologous proteins activity, or to achieve regulation of gene function. Such technology is now well know in the art, and sense or antisense oligomers or larger fragments, can be designed from various locations along the coding or control regions of sequences encoding CG3842 homologous proteins Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the gene encoding CG3842 homologous proteins. These techniques are described both in Sambrook et al. (supra) and in Ausubel et al. (supra). Genes encoding CG3842 homologous proteins can be turned off by transforming a cell or tissue with expression vectors which express high levels of polynucleotide or fragment thereof which encodes CG3842 homologous proteins. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, DNA, RNA, or nucleic acid analogues such as PNA, to the control regions of the gene encoding CG3842 homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyse endonucleolytic cleavage of sequences encoding CG3842 homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding CG3842 homologous proteins. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of CG3842 homologous proteins, antibodies to CG3842 homologous proteins, mimetics, agonists, antagonists, or inhibitors of CG3842 homologous proteins. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. After pharmaceutical compositions have been prepared, they can be placed in an appropriate container and labeled for treatment of an indicated condition. For administration of CG3842 homologous proteins, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example CG3842 nucleic acids or proteins or fragments thereof, or antibodies, which is sufficient for treating a specific condition. Therapeutic efficacy of homologous proteins and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combinations), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind CG3842 homologous proteins may be used for the diagnosis of conditions or diseases characterized by or associated with over- or underexpression of CG3842, or in assays to monitor patients being treated with CG3842 homologous proteins, agonists, antagonists or inhibitors. The antibodies useful for diagnostic purposes may be prepared in the same manner as those described above for therapeutics. Diagnostic assays for CG3842 homologous proteins include methods, which utilize the antibody and a label to detect CG3842 homologous proteins in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring CG3842 homologous proteins are known in the art and provide a basis for diagnosing altered or abnormal levels of CG3842 homologous proteins expression. Normal or standard values for CG3842 homologous proteins expression are established by combining body fluids or cell extracts taken from normal mammalian subjects, preferably human, with antibody to CG3842 homologous proteins under conditions suitable for complex formation. The amount of standard complex formation may be quantified by various methods, but preferably by photometric means. Quantities of CG3842 homologous proteins expressed in control and disease, samples from e.g. biopsied tissues are compared with the standard values. Deviation between standard and subject values establishes the parameters for diagnosing disease.

In another embodiment of the invention, the polynucleotides specific for CG3842 homologous proteins may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of CG3842 homologous proteins may be correlated with disease. The diagnostic assay may be used to distinguish between absence, presence, and excess expression of CG3842 homologous proteins, and to monitor regulation of CG3842 homologous proteins levels during therapeutic intervention.

In one aspect, hybridization with PCR probes which are capable of detecting polynucleotide sequences, including genomic sequences, encoding CG3842 homologous proteins closely related molecules, may be used to identify nucleic acid sequences which encode CG3842 homologous proteins. The specificity of the probe, whether it is made from a highly specific region, e.g., unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding CG3842 homologous proteins, alleles, or related sequences. Probes may also be used for the detection of related sequences, and should preferably contain at least 50% of the nucleotides from any of the CG3842 homologous proteins encoding sequences. The hybridization probes of the subject invention may be DNA or RNA and derived from the nucleotide sequence of the polynucleotide comprising the nucleic acid sequence of nucleic acids encoding a Drosophila protein (GadFly Accession Number CG3842) or human homologous proteins, or from a genomic sequence including promoter, enhancer elements, and introns of the naturally occurring CG3842 homologous proteins. Means for producing specific hybridization probes for DNAs encoding CG3842 homologous proteins include the cloning of nucleic acid sequences encoding CG3842 homologous proteins derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences encoding CG3842 homologous proteins may be used for the diagnosis of conditions or diseases, which are associated with expression of CG3842 homologous proteins. Examples of such conditions or diseases include, but are not limited to, metabolic diseases and disorders, including obesity and diabetes. Polynucleotide sequences specific for CG3842 homologous proteins may also be used to monitor the progress of patients receiving treatment for metabolic diseases and disorders, including obesity and diabetes. The polynucleotide sequences specific for CG3842 homologous proteins may be used in Southern or Northern analysis, dot blot, or other membrane-based technologies; in PCR technologies; or in dip stick, pin, ELISA or chip assays utilizing fluids or tissues from patient biopsies to detect altered CG3842 homologous proteins expression. Such qualitative or quantitative methods are well known in the art.

In a particular aspect, the nucleotide sequences specific for CG3842 homolog proteins may be useful in assays that detect activation or induction of various metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and/or gallstones. The nucleotide sequences encoding CG3842 homolog proteins may be labeled by standard methods, and added to a fluid or tissue sample from a patient under conditions suitable for the formation of hybridization complexes. After a suitable incubation period, the sample is washed and the signal is quantitated and compared with a standard value. The presence of altered levels of nucleotide sequences encoding CG3842 homolog proteins in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of CG3842 homolog proteins, a normal or standard profile for expression is established. This may be accomplished by combining body fluids or cell extracts taken from normal subjects, either animal or human, with a sequence which encodes CG3842 homolog proteins or a fragment thereof, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases (metabolic syndrome), the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders. Additional diagnostic uses for oligonucleotides designed from the sequences encoding CG3842 homolog proteins may involve the use of PCR. Such oligomers may be chemically synthesized, generated enzymatically, or produced from a recombinant source. Oligomers will preferably consist of two nucleotide sequences, one with sense orientation and another with antisense, employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of CG3842 homolog proteins include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantification of multiple samples may be accelerated by running the assay in an ELISA format where the oligomer of interest is presented in various dilutions and a spectrophotometric or calorimetric response gives rapid quantification.

In another embodiment of the invention, the nucleic acid sequences, which encode CG3842 homolog proteins, may also be used to generate hybridization probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding CG3842 on a physical chromosomal map and a specific disease, or predisposition to a specific disease, may help to delimit the region of DNA associated with that genetic disease.

The nucleotide sequences of the CG3842 homolog proteins may be used to detect differences in gene sequences between normal, carrier, or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11 q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, the CG3842 homolog proteins, their catalytic or immunogenic fragments or oligopeptides thereof, an in vitro model, a genetically altered cell, or animal, can be used for screening libraries of compounds in any of a variety of drug screening techniques.

One can identify effectors, e.g. receptors, enzymes, proteins, ligands, or substrates that bind to, modulate or mimic the action of one or more of the proteins of the invention. The protein or a fragment thereof employed in such screening may be free in solution, affixed to a solid support, borne on a cell surface, or located intracellularly. The formation of binding complexes, between CG3842 homolog proteins and the agent tested, may be measured. Agents could, either directly or indirectly, influence the activity of the proteins of the invention. Mechanisms of direct influence could for example, but not exclusively, be the interference of agents with either substrate or cofactor recognition, binding, and conversion. For example the dehydrogenase activity could be measured in vitro by using recombinantly expressed and purified CG3842 homolog proteins or fragments thereof.

For example, an in vitro measurement of the oxidoreductase activity of the proteins of the invention can be performed by using tritiated steroids, as for example but not limited to dihydrotestosterone, progesterone, corticosterone, aldosterone, androsterone, allopregnanolone, or 3-androstanediol. The reaction can be driven in the oxidative direction in the presence of the cofactors NADP⁺/NAD⁺ or in the reductive direction in the presence of NADPH/NADH. The reaction products can be extracted and analysed on silica gel TLC plates. Alternatively, retinoid substrates, i.e. all-trans and cis-isomers of retinoids, can be used as oxidoreductase substrates and product generation can be analysed by procedures known in the art, such as HPLC separation (Kedishvili et al. (2002), J. Biol. Chem. 277 (No. 32), 28909-28915). In another embodiment of the invention, the oxidoreductase activity of the enzymes can be monitored following the change in fluorescence by conversion of NADPH or NADH to or from NADP+ or NAD+, respectively.

In addition, activity of CG3842 homolog proteins against its physiological substrate(s) or derivatives thereof can be measured in cell-based assays.

Agents may also interfere with posttranslational modifications of the protein, such as phosphorylation and dephosphorylation, farnesylation, palmitoylation, acetylation, alkylation, ubiquitination, proteolytic processing, subcellular localization, and degradation. Moreover, agents can influence the dimerization or oligomerization of the proteins of the invention or, in a heterologous manner, of the proteins of the invention with other proteins, for example, but not exclusively, docking proteins, enzymes, receptors, or translation factors. Agents can also act on the physical interaction of the proteins of this invention with other proteins, which are required for protein function, for example, but not exclusively, their downstream signaling.

Methods for determining protein-protein interaction are well known in the art. For example, binding of a fluorescently labeled peptide derived from CG3842 homolog protein interacting protein (or vice versa), can be detected by a change in polarisation. In case that both binding partners, which can be either the full length proteins as well fragments thereof are fluorescently labeled, binding can be detected by fluorescence energy transfer (FRET) from one fluorophore to the other. In addition, a variety of commercially available assay principles suitable for detection of protein-protein interaction are well known in the art, for example but not exclusively AlphaScreen (PerkinElmer) or Scintillation Proximity Assays (SPA) by Amersham. Alternatively, the interaction of CG3842 homolog proteins with cellular proteins can be the basis for a cell-based screening assay, in which both proteins are fluorescently labeled and interaction of both proteins is detected by analyzing cotranslocation of both proteins with a cellular imaging reader, as developed for example, but not exclusively, by Cellomics or EvotecOAI. In all cases, the two or more binding partners can be different proteins with one being a CG3842 homolog protein, or in case of dimerization and/or oligomerization the CG3842 homolog protein itself. CG3842 homolog proteins, for which one target mechanism of interest, but not the only one, would be such protein/protein interactions are unnamed human protein XP_{—}085058 and human CGI-82.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to CG3842 homolog proteins, large numbers of different small test compounds are synthesized on a solid substrate, such as plastic pins or some other surface. The test compounds are reacted with CG3842 homolog proteins, or fragments thereof, and washed. Bound CG3842 homolog proteins are then detected by methods well known in the art. Purified CG3842 homolog proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding CG3842 homolog proteins specifically compete with a test compound for binding CG3842 homolog proteins. In this manner, the antibodies can be used to detect the presence of any peptide, which shares one or more antigenic determinants with CG3842 homolog proteins. In additional embodiments, the nucleotide sequences which encode CG3842 homolog proteins may be used in any molecular biology techniques that have yet to be developed, provided the new techniques rely on properties of nucleotide that are currently known, including, but not limited to, such properties as the triplet genetic code and specific base pair interactions.

The nucleic acids encoding the proteins of the invention can be used to generate transgenic cell lines and animals. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounts can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al, 1998, Mol. Cell. 2:449-569). Susceptible wild type mice (for example C57BI/6) show similiar symptoms if fed a high fat diet. In addition to testing the expression of the proteins of the invention in such mouse strains (see EXAMPLES), these mice could be used to test whether administration of a candidate modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a SCAD nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

The kit may be used for diagnostic or therapeutic purposes or for screening applications as described above. The kit may further contain user instructions.

The FIGURES show:

FIG. 1 shows the increase of triglyceride content of PX2287.1 flies (referred to as “2287.1”) caused by integration of the P-vector (in comparison to controls with integration of these vectors elsewhere in genome, referred to as “TG010419, n=60”).

FIG. 2 shows the molecular organization of the mutated gene locus of Gadfly Accession Number CG3842.

FIG. 3 shows the BLASTP search results for Gadfly Accession Number CG3842 (Query) with the best human homologous matches (Sbject).

FIG. 3A shows the homology to human unnamed protein with GenBank Accession Number XP_{—}085058.1 (SEQ ID NO: 2)

FIG. 3B shows the homology to human PAN2 protein (GenBank Accession Number NP_{—}065956.1).

FIG. 3C shows the homology to human CGI-82 protein (GenBank Accession Number NP_{—}057110.1, SEQ ID NO: 4).

FIG. 4 shows the Clustal X (1.81) multiple sequence alignment analysis containing protein sequences for human CGI-82 (Accession Number NP_{—}057110, SEQ ID NO: 4), human XP_{—}085058 (SEQ ID NO: 2), Drosophila GadFly Accession Number CG3842, and human PAN2 (Accession Number NP_{—}065956)

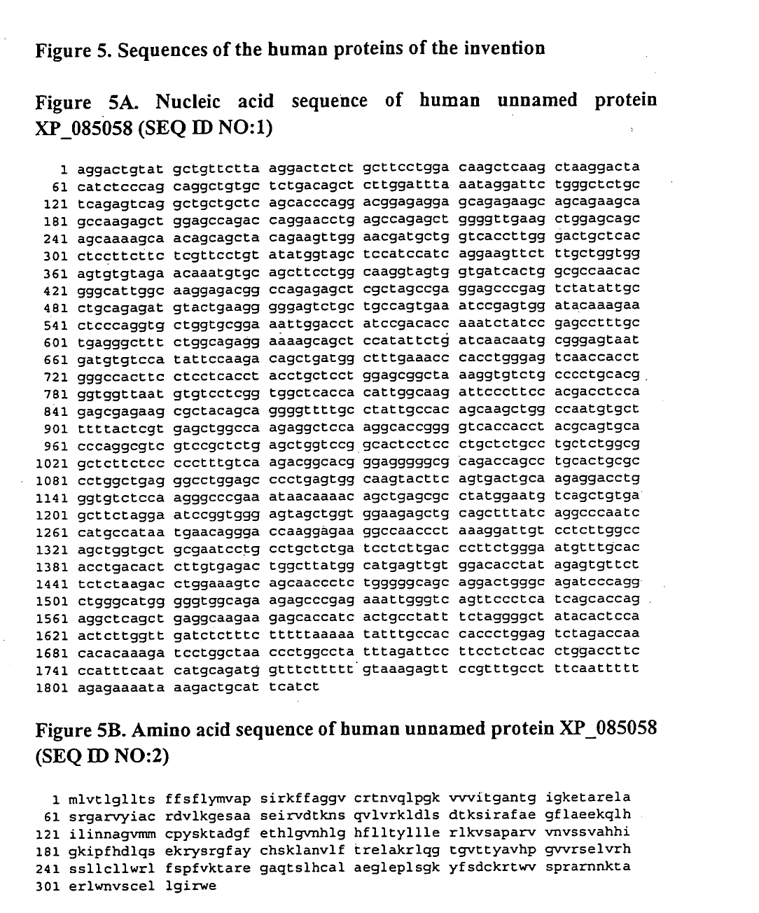

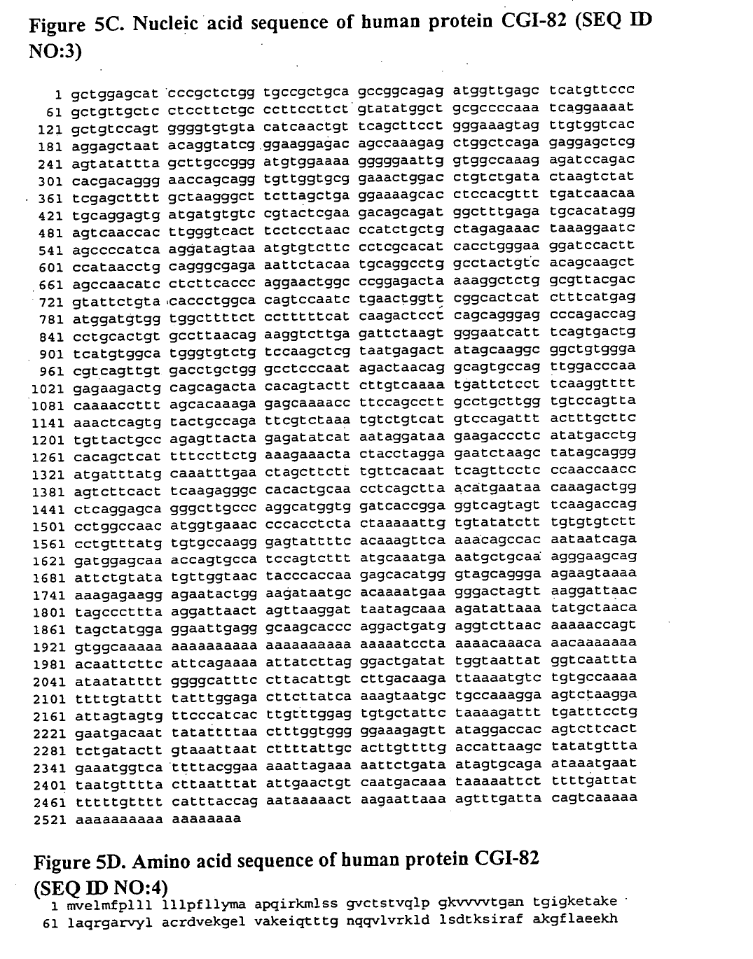

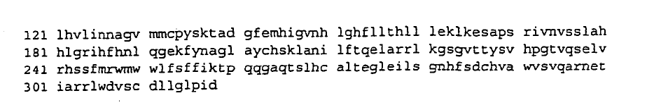

FIG. 5 shows the human sequences of the invention

FIG. 5A. shows the nucleic acid sequence of human unnamed protein XP_{—}085058 (also referred to herein as DG21-1) (SEQ ID NO:1; GenBank Accession Number XM_{—}085058).

FIG. 5B. shows the amino acid sequence of human unnamed protein XP_{—}085058 (SEQ ID NO:2; GenBank Accession Number XP_{—}085058).

FIG. 5C. shows the nucleic acid sequence of human CGI-82 (also referred to herein as DG21-2 or PSDR1) (SEQ ID NO:3; GenBank Accession Number NM_{—}016026).

FIG. 5D. shows the amino acid sequence of human CGI-82 (also referred to herein as DG21-2 or PSDR1) (SEQ ID NO:4; GenBank Accession Number NP_{—}057110).

FIG. 6. Expression of unnamed protein (DG21-1) in mammalian tissues. The relative RNA-expression is shown on the X-axis. In FIGS. 6A, B, and C, the tissues tested are given on the X-axis. “WAT” refers to white adipose tissue, “BAT” refers to brown adipose tissue. In FIGS. 6D, E, and F, the X-axis represents the time axis. “d0” refers to day 0 (start of the experiment), “d2”-“d10” refers to day 2—day 10 of adipocyte differentiation).

FIG. 6A: Real-time PCR analysis of unnamed protein (DG21-1) in wildtype mouse tissues.

FIG. 6B: Real-time PCR mediated analysis of unnamed protein (DG21-1) in different mouse models.

FIG. 6C: Real-time PCR mediated analysis of unnamed protein (DG21-1) in different mouse models (different diets).

FIG. 6D: Real-time PCR mediated analysis of unnamed protein (DG21-1) expression during the differentiation of 3T3-L1 cells from preadipocytes to mature adipocytes.

FIG. 6E: Real-time PCR mediated analysis of unnamed protein (DG21-1) expression during the differentiation of 3T3-F442A cells from preadipocytes to mature adipocytes.

FIG. 6F: Real-time PCR mediated analysis of unnamed protein (DG21-1) expression during the differentiation of TA1 cells from preadipocytes to mature adipocytes.

FIG. 7. Expression of CGI-82 (DG21-2) in mammalian tissues. The relative RNA-expression is shown on the Y-axis. The tissues tested are given on the X-axis. “WAT” refers to white adipose tissue, “BAT” refers to brown adipose tissue.

FIG. 7A: Real-time PCR analysis of CGI-82 (DG21-2) in wildtype mouse tissues.

FIG. 7B: Real-time PCR mediated analysis of CGI-82 (DG21-2) in different mouse models.

FIG. 7C: Real-time PCR mediated analysis of CGI-82 (DG21-2) in different mouse models (different diets).

FIG. 8 shows in vitro assays for the determination of glycogen, fatty acid metabolism and lipid synthesis in cells overexpressing unnamed protein (DG21-1) or CGI-82 (DG21-2)

FIG. 8A shows an increase in glycogen levels in cells overexpressing unnamed protein (DG21-1) versus control cells. The Y-axis shows cellular glycogen levels (μg/mg protein) and the X-axis shows days of cell differentiation.

FIG. 8B shows no changes in glycogen levels in cells overexpressing CGI-82 (DG21-2) versus control cells. The Y-axis shows cellular glycogen levels (μg/mg protein) and the X-axis shows days of cell differentiation.

FIG. 8C shows an increase in free fatty acid uptake in cells overexpressing unnamed protein (DG21-1) or CGI-82 (DG21-2). The Y-axis shows free fatty acid levels (shown as DPM per mg protein).

FIG. 8D shows a reduction in free fatty acid esterification in cells overexpressing unnamed protein (DG21-1) or CGI-82 (DG21-2). The Y-axis shows free fatty acid levels (shown as DPM per mg protein).

FIG. 8E shows an increase in lipid synthesis in cells overexpressing unnamed protein (DG21-1) or CGI-82 (DG21-2) with or without insulin stimulation. The Y-axis shows lipid synthesis (shown as DPM per mg protein).

The examples illustrate the invention:

### EXAMPLE 1

### Measurement of Energy Storage Metabolites Content

Mutant flies are obtained from a proprietary fly mutation stock collection. The flies are grown under standard conditions known to those skilled in the art. In the course of the experiment, additional feedings with bakers yeast (Saccharomyces cerevisiae) are provided. The average increase of triglyceride content of Drosophila flies containing the transposon vector in the homozygous viable PX2287.1 integration was investigated in comparison to control flies (FIG. 1). For determination of triglyceride content, flies were incubated for 5 min at 70° C. in an aqueous buffer using a waterbath, followed by hot extraction. After another 5 min incubation at 70° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol. The assay was repeated several times. The average triglyceride level of all flies of the PX collection is shown as 100% in FIG. 1. PX2287.1 homozygous flies show constantly a higher triglyceride content than the controls The average increase of triglyceride content of the homozygous viable Drosophila line PX2287.1 is 80% (column 2 in FIG. 1). Therefore, the change of gene activity in the locus of the PX2287.1 integration on chromosome X where the EP-vector of PX2287.1 flies is homozygous viable integrated, is responsible for changes in the metabolism of the energy storage triglycerides.

### EXAMPLE 2

### Identification of a Drosophila Gene Associated with Metabolic Control

In FIG. 2, genomic DNA is represented by the assembly as a thin black line in the middle (numbers represent the length in basepairs of the genomic DNA) that includes the integration sites of vector for line PX2287.1. Transcribed DNA sequences (ESTs) and predicted exons are shown as bars on the two sides (sense and antisense strand). Predicted exons of the cDNA are shown as dark grey bars and introns as light grey lines. The sequence encodes for a gene that is predicted by GadFly sequence analysis programs as Accession Number CG3842. Using those isolated genomic sequences public databases like Berkeley Drosophila Genome Project (GadFly) were screened confirming the homozygous viable integration site of the PX2287.1 vector 542 basepairs downstream of the coding sequence of CG3842, causing an increase of triglyceride content (the site of integration is shown as vertical dotted line). Therefore, expression of the cDNA encoding Accession Number CG3842 could be effected by homozygous viable integration of vectors of line PX2287.1, leading to an increase of the energy storage triglycerides.

### EXAMPLE 3

### Identification of Human CG3842 Homologues

CG3842 homologous proteins and nucleic acid molecules coding therefore are obtainable from insect or vertebrate species, e.g. mammals or birds. The most similar human nucleic acid sequences and the proteins encoded thereby have been determined using the BLAST algorithm searching public GenBank databases (see FIG. 3). The most homologous human proteins are unnamed protein with GenBank Accession Number XP_{—}085058 (64% homology; see FIG. 3A), human CGI-82 protein (GenBank Accession Number NM_{—}016026; 62% homology; see FIG. 3C), and PAN2 (GenBank Accession Number NM_{—}020905; 59% homology; see FIG. 3B).

The results of a comparison of the Drosophila adh-short domains (e.g. GadFly Accession Number CG3842, amino acids 73 to 328) with the adh-short domains unnamed protein with GenBank Accession Number XP_{—}085058, of human CGI-82 protein (GenBank Accession Number NM_{—}016026), and of human PAN2 protein (GenBank Accession Number NM_{—}020905) in a pairwise alignment are shown in TABLE 1.

TABLE 1
Results of pairwise alignment of deduced amino
acid sequences of Drosophila GadFly Accession
Numbers CG3842 and closely related human proteins
unnamed protein XP_085058, CGI-82 (PSDR1), and PAN2.

| Human protein | Drosophila protein | Identities/Similarities |
|---|---|---|
| XP_085058 | CG3842 | 56%/67% |
| CGI-82 | CG3842 | 55%/66% |
| PAN2 | CG3842 | 52%/63% |

A ClustaW (1.81) multiple sequence alignment has been conducted among the adh-short domains of the proteins described in TABLE 1 above and is shown in FIG. 4.

### EXAMPLE 4

### Expression of the Polypeptides in Mammalian Tissues

To analyse the expression of the mRNA coding for the proteins of the invention in mammalian tissues, several mouse strains (preferably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferably 22° C.), 40 percent humidity and a light/dark cycle of preferably 14/10 hours. The mice were fed a standard chow (for example, from ssniff Spezialitäten GmbH, order number ssniff M-Z V1126-000). For the fasting experiment, wild type mice were starved for 48 h without food, but only water supplied ad libitum. (see, for example, Schnetzler et al. J Clin Invest 1993 July;92(1):272-80, Mizuno et al. Proc Natl Acad Sci USA 1996 Apr. 16;93(8):3434-8). Animals were sacrificed at an age of 6 to 8 weeks. The animal tissues were isolated according to standard procedures known to those skilled in the art, snap frozen in liquid nitrogen and stored at −80° C. until needed.

To analyse the role of the proteins disclosed in this invention in the in vitro differentiation of different mammalian cell culture cells from pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC—CL 173). Alternatively, a mammalian fibroblast TA1 cell line, a murine preadipocyte line derived from T101/2 mouse embryo fibroblasts (Chapman et al., 1984, J Biol Chem 259(24):15548-55) or a 3T3 derived celline called 3T3-F442A was used. 3T3-L1 or TA-1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). In brief, cells were plated in DMEM/10% FCS (Invitrogen, Karlsruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/HamF12 (3:1; Invitrogen), Fetuin (300 microg/ml; Sigma, Munich, Germany), Transferrin (2 microg/ml; Sigma), pantothenate (17 microM; Sigma), biotin (1 microM; Sigma), and EGF (0.8 nM; Hoffmann-La Roche, Basel, Switzerland). Differentiation was induced by adding Dexamethasone (DEX; 0.1 μM; Sigma), 3-methyl-isobutyl-1-methylxanthine (MIX; 0.5 mM; Sigma), and bovine insulin (5 μg/ml; Invitrogen) for four days. Four days after confluence (d4), cells were kept in SF medium until differentiation was completed at d12. The medium was changed every 48 hours and suitable aliquots of cells were taken.

TaqMan Analysis of the RNA Expression

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferably using Superscript II RNaseH-Reverse Transcriptase, from Invitrogen, Karlsruhe, Germany) and subjected to Taqman analysis preferably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

The following primer/probe pairs were used for the TaqMan analysis (GenBank Accession Number AK020927 for mouse DG-21-1 sequence homolog to human unnamed protein XP_{—}085058; GenBank Accession Number AB030503 for mouse DG-21-2 sequence, homolog to human protein CGI-82):

(SEQ ID NO: 5)

Mouse DG-21-1 forward primer:
5′-CAAGGGCCCGGAACAAG-3′;

(SEQ ID NO: 6)

Mouse DG-21-1 reverse primer:
5′-TGGATTCCTAGAAGCTCGCAG-3′;

(SEQ ID NO: 7)

Mouse DG-21-1 Taqman probe (5/6-FAM):
AACAGCTGAGCGATTGTGGAACGTCA (5/6-TAMRA)

(SEQ ID NO: 8)

Mouse DG-21-2 forward primer:
5′-AATCCAGGACTACATTTCCCAGAG-3′;

(SEQ ID NO: 9)

Mouse DG-21-2 reverse primer:
5′-CCTAGACACACCCCGGTTCA-3′;

(SEQ ID NO: 10)

Mouse DG-21-2 Taqman probe (5/6-FAM):
CTTTGCTCCCTGGCGGTTGCAG (5/6-TAMRA)

The expression profiling studies confirm the particular relevance of the proteins of the invention as regulators of energy metabolism in mammalian cells. Taqman analysis revealed that the proteins of the invention are ubiquitously expressed in different types of mamalian tissues including metabolic active tissues such as white (WAT) and brown (BAT) adipose tissue. However, DG21-1 mRNA is predominantly expressed in spleen, bone marrow, lung, WAT and muscle tissues compared to other tissue types in wild type mouse as depicted in FIG. 6A. DG21-2, although also ubiqutously expressed, shows an highest expression in testis (FIG. 7A).

Further, we show in this invention that the transcription of the genes coding for the proteins of the invention is regulated by fasting, high fat diet (HFD), or by genetically induced obesity. In this invention, we used mouse models of insulin resistance and/or diabetes, such as mice carrying gene knockouts in the leptin pathway (for example, ob (leptin) or db (leptin receptor/ligand) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569).

We found in this invention that DG21-1 (unnamed protein) mRNA is upregulated in pancreas and metabolic active tissue brown adipose tissue (BAT) of fasted and in BAT of genetically induced obese mice (ob-ob) compared to wildtype (wt) mice (FIG. 6B) and also in pancreas and BAT of susceptible wild type mice (for example C57BI/6) that show symptoms of diabetes, lipid accumulation, and high plasma lipid levels, if fed a high fat diet (HFD) (FIG. 6C). DG-21-2 (homolog to CGI-82=PSDR1) is strongly up-regulated in white adipose tissue (WAT) of ob/ob mice compared to wt mice (FIG. 7B) and also in BAT and muscle of mice fed a HFD compared to mice fed a standard diet (FIG. 7C).

Unnamed protein XP_{—}085058 (DG21-1) is significantly up regulated (35 fold up regulation on day 6) during differentiation of 3T3-L1 cells (FIG. 6E). as well as in 3T3-F442A cells (60 fold upregulation on day 4) (FIG. 6D) and TA1 cells (30 fold upregulation on day 12) cells (see FIG. 6F).

The tissue specific regulation of unnamed protein XP_{—}085058 (DG-21-1) and CGI-82 (DG-21-2) and the regulation of DG-21-1 during in vitro adipocyte differentiation shows clearly that the proteins of the invention are modulatorsof adipogenesis and metabolism in mammalian cells.

### EXAMPLE 5

### In Vitro Assays in Cells Overexpressing the Proteins of the Invention

Obesity is caused by an inbalance of energy stored and energy used by the regarding organism. In mammalian cells energy is stored by energy storage metabolites (ESM), mainly as glycogen or triglycerides. Glycogen is used as a quick response to urgent energy needs whereas triglycerides are used as fuel for long term energy expenditure. In this invention, we therefore analyzed the level of energy storage metabolites in cells overexpressing the proteins of the invention.

Retroviral Infection of Preadipocytes

Packaging cells were transfected with retroviral plasmids pLPCX carrying the mouse genes coding for the proteins of the invention and a selection marker using calcium phosphate procedure. Control cells were infected with pLPCX carrying no transgene. Briefly, exponentially growing packaging cells were seeded at a density of 350,000 cells per 6-well in 2 ml DMEM+10% FCS one day before transfection. 10 min before transfection chloroquine was added directly to the overlying medium (25 microM end concentration). A 250 μl transfection mix consisting of 5 μg plasmid-DNA (candidate:helper-virus in a 1:1 ratio) and 250 mM CaCl₂ was prepared in a 15 ml plastic tube. The same volume of 2×HBS (280 μM NaCl, 50 μM HEPES, 1.5 mM Na₂HPO₄, pH 7.06) was added and air bubbles were injected into the mixture for 15 sec. The transfection mix was added drop wise to the packaging cells, distributed and the cells were incubated at 37° C., 5% CO₂ for 6 hours. The cells were washed with PBS and the medium was exchanged with 2 ml DMEM+10% CS per 6-well. One day after transfection the cells were washed again and incubated for 2 days of virus collection in 1 ml DMEM+10% CS per 6-well at 32° C., 5% CO₂. The supernatant was then filtered through a 0.45 μm cellulose acetate filter and polybrene (end concentration 8 μg/ml) was added. Mammalian fibroblast (3T3-L1) cells in a sub-confluent state were overlaid with the prepared virus containing medium. The infected cells were selected for 1 week with 2 μg/ml puromycin. Following selection the cells were checked for transgene expression by western blot and immunofluorescence. Overexpressing cells were seeded for differentiation. 3T3-L1 cells were maintained as fibroblasts and differentiated into adipocytes as described in the prior art and above. For analysing the role of the proteins disclosed in this invention in vitro assays for the determination of ESM storage, synthesis and transport were performed.

Preparation of Cell Lysates for Analysis of Energy Storage Metabolites

Starting at confluence (d0), cell media was changed every 48 hours. Cells were harvested 6 hours after media change as follows. Media was collected, and cells were washed twice in PBS prior to lyses in 600 μl HB-buffer (0.5% polyoxyethylene 10 tridecylethan, 1 mM EDTA, 0.01 M NaH₂PO₄, pH 7.4). After inactivation at 70° C. for 5 minutes, cell lysates were prepared on Bio 101 systems lysing matrix B (0.1 mm silica beads; Q-Biogene, Carlsbad, USA) by agitation for 2×45 seconds at a speed of 4.5 (Fastprep FP120, Bio 101 Thermosavant, Holbrock, USA). Supernatants of lysed cells were collected after centrifugation at 3000 rpm for 2 minutes, and stored in aliquots for later analysis at −80° C.

Changes in Cellular Glycogen Levels During Adipogenesis

Cell lysates were analysed in duplicates in 96-well plates for total protein and glycogen content using the Bio-Rad DC Protein assay reagent (Bio-Rad, Munich, Germany) according to the manufacturer's instructions and an enzymatic starch kit from Hoffmann-La Roche (Basel, Switzerland). 10-μL samples were incubated with 20-μl amyloglucosidase solution for 15 minutes at 60° C. to digest glycogen to glucose. The glucose is further metabolised with 100 μl distilled water and 100 μl of enzyme cofactor buffer and 12 μl of enzyme buffer (hexokinase and glucose phosphate dehydrogenase). Background glucose levels are determined by subtracting values from a duplicate plate without the amyloglucosidase. Final absorbance is determined at 340 nm. HB-buffer as blank, and a standard curve of glycogen (Hoffmann-La Roche) were included in each assay. Glycogen content in samples were calculated using a standard curve.

Glucose is taken up by the cells rapidly and stored in the form of glycogen. This energy storage is than primarily used for the metabolic demands of the cell. The Cellular glycogen levels were increased throughout adipogenesis (beginning on d6 with a maximum on d8 lasting up to d12) in differentiated adipocytes overexpressing unnamed protein XP_{—}085058, as shown in FIG. 8A. This increase in glycogen level as a consequence of over expression of unnamed protein XP_{—}085058 could reflect an elevated glucose uptake, a higher glycogen synthesis rate or a decreased energy consumption and thus confirms a role of unnamed protein XP_{—}085058 in metabolic regulation.

Synthesis of Lipids During Adipogenesis

During the terminal stage of adipogenesis (d12) cells were analysed for their ability to metabolise lipids. A modified protocol to the method of Jensen et al. (2000) for lipid synthesis was established. Cells were washed 3 times with PBS prior to serum starvation in Krebs-Ringer-Bicarbonate-Hepes buffer (KRBH; 134 nM NaCl, 3.5 mM KCl, 1.2 mM KH₂ PO₄, 0.5 mM MgSO₄, 1.5 mM CaCl₂, 5 mM NaHCO₃, 10 mM Hepes, pH 7.4), supplemented with 0.1% FCS for 2.5 h at 37° C. For insulin-stimulated lipid synthesis, cells were incubated with 1 μM bovine insulin (Sigma; carrier: 0.005N HCl) for 45 min at 37° C. Basal lipid synthesis was determined with carrier only. ¹⁴C(U)-D-glucose NEN Life Sciences) in a final activity of 1 μCi/Well/ml in the presence of 5 mM glucose was added for 30 min at 37° C. For the calculation of background radioactivity, 25 μM cytochalasin B (Sigma) was used. All assays were performed in duplicate wells. To terminate the reaction, cells were washed 3 times with ice cold PBS, and lysed in 1 ml 0.1 N NaOH. Protein concentration of each well was assessed using the standard Biuret method (Protein assay reagent; Bio-Rad). Total lipids were separated from aqueous phase after overnight extraction in Insta-Fluor scintillation cocktail (Packard Bioscience) followed by scintillation counting.

We show in this invention that cells overexpressing unnamed protein XP_{—}085058 are more effective in synthesising lipids from exogenous glucose. Consequently, the levels of insulin stimulated lipid synthesis of cells overexpressing unnamed protein XP_{—}085058 are also higher at day 12 of adipogenesis when compared to control cells (FIG. 8E). On the same day of differentiation, the lipid synthesis of CGI-82 overexpressing cells can not be stimulatd by insulin as with the control cells (FIG. 8E). The increased level of lipid synthesis from exogenous glucose in cells overexpressing unnamed protein XP_{—}085058 demonstrate an activation of enzymes involved in lipid synthesis in these cells as compared to control cells.

Transport and Metabolism of Free Fatty Acids During Adipogenesis (FIG. 8C)

During the terminal stage of adipogenesis (d12) cells were analysed for their ability to transport long chain fatty acid across the plasma membrane. A modified protocol to the method of Abumrad et al (1991)(Proc. Natl. Acad. Sci. USA, 1991: 88; 6008-12) for cellular transportation of fatty acid was established. In summary, cells were washed 3 times with PBS prior to serum starvation. This was followed by incubation in KRBH buffer supplemented with 0.1% FCS for 2.5h at 37° C. Uptake of exogenous free fatty acids was initiated by the addition of isotopic media containing non radioactive oleate and (³H)oleate (NEN Life Sciences) complexed to serum albumin in a final activity of 1 μCi/Well/ml in the presence of 5 mM glucose for 30 min at room temperature (RT). For the calculation of passive diffusion (PD) in the absence of active transport (AT) across the plasma membrane 20 mM of phloretin in glucose free media (Sigma) was added for 30 min at RT. All assays were performed in triplicate wells. To terminate the active transport 20 mM of phloretin in glucose free media was added to the cells. Cells were lysed in 1 ml 0.1N NaOH and the protein concentration of each well were assessed using the standard Biuret method (Protein assay reagent; Bio-Rad). Esterified fatty acids were separated from free fatty acids using overnight extraction in Insta-Fluor scintillation cocktail (Packard Bioscience) followed by scintillation counting.

We found in this invention that transport of exogenous fatty acids across the plasma membrane of overexpressing cells overexpressing unnamed protein XP_{—}085058 were significantly higher at day 12 of adipogenesis when compared to control cells (FIG. 8C). We also show in this invention that esterification of fatty acids of CGI-82 overexpressing cells but not cells overexpressing unnamed protein XP_{—}085058 were significantly lower at day 12 of adipogenesis when compared to control cells (FIG. 8D).

The profound increase in uptake of free fatty acids of cells overexpressing unnamed protein XP_{—}085058 and the decrease of the free fatty acid esterification in CGI-82 overexpressing cells could be due to a direct action of the proteins of the invention or alternatively due to a role in the regulation of free fatty acid uptake and esterification. The proteins of the invention may for example play a role in converting regulatory inactive retinoids or steroids in regulatory activ hormone derivatives which then influence fatty acid metabolism directly or regulate the gene expression of fatty acid metabolic enzymes or transporters on the transcriptional level.

### EXAMPLE 6

### Generation and Analysis of Transgenic Mice

Generation of the Transgenic Animals

Mouse cDNA was isolated from mouse brown adipose tissue (BAT) using standard protocols as known to those skilled in the art. The cDNA was amplified by RT-PCR and point mutations were introduced into the cDNA. The resulting mutated cDNA was cloned into a suitable transgenic expression vector. The transgene was microinjected into the male pronucleus of fertilized mouse embryos (preferably strain C57/BL6/CBA F1 (Harlan Winkelmann). Injected embryos were transferred into pseudo-pregnant foster mice. Transgenic founders were detected by PCR analysis. Two independent transgenic mouse lines containing the construct were established and kept on a C57/BL6 background. Briefly, founder animals were backcrossed with C57/BL6 mice to generate F1 mice for analysis. Transgenic mice were continously bred onto the C57/BI6 background. The expression of the proteins of the invention can be analyzed by taqman analysis as described above, and further analysis of the mice can be done as known to those skilled in the art.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a nucleic acid molecule of the short-chain dehydrogenase (SCAD) gene family or a polypeptide encoded thereby or a fragment or a variant of said nucleic acid molecule or said polypeptide or an effector of a nucleic acid molecule of the SCAD gene family or said polypeptide, e.g. an antibody, an aptamer or another receptor recognizing a nucleic acid molecule of the SCAD gene family or said polypeptide encoded thereby, preferably together with pharmaceutically acceptable carriers, diluents and/or adjuvants.

2. The composition of claim 1, wherein the nucleic acid molecule is a vertebrate or insect SCAD nucleic acid, particularly a nucleic acid encoding a Drosophila protein (GadFly Accession Number CG3842), an unnamed protein (SEQ ID NO: 1 and 2), a human CGI-82 protein (SEQ ID NO: 3 and 4), or or a human PAN2 protein (GenBank Accession Number NP_{—}065956 for the protein, NM_{—}020905 for the cDNA) or a fragment there of or a variant thereof and/or a nucleic acid complementary thereto.

3. The composition of claim 1 or 2, wherein said nucleic acid molecule
(a) hybridizes at 50° C. in a solution containing 1×SSC and 0.1% SDS to a nucleic acid molecule as defined in claim 2 and/or a nucleic acid molecule which is complementary thereto;
(b) it is degenerate with respect to the nucleic acid molecule of (a)
(c) encodes a polypeptide which is at least 85%, preferably at least 90%, more preferably at least 95%, more preferably at least 98% and up to 99,6% identical to a SCAD polypeptide as defined in claim 2;
(d) differs from the nucleic acid molecule of (a) to (c) by mutation and wherein said mutation causes an alteration, deletion, duplication or premature stop in the encoded polypeptide.

4. The composition of any one of claims 1-3, wherein the nucleic acid molecule is a DNA molecule, particularly a cDNA or a genomic DNA.

5. The composition of any one of claims 1-4, wherein said nucleic acid encodes a polypeptide contributing to regulating the energy homeostasis and/or the metabolism of triglycerides.

6. The composition of any one of claims 1-5, wherein said nucleic acid molecule is a recombinant nucleic acid molecule.

7. The composition of any one of claims 1-6, wherein the nucleic acid molecule is a vector, particularly an expression vector.

8. The composition of any one of claims 1-5, wherein the polypeptide is a recombinant polypeptide.

9. The composition of claim 8, wherein said recombinant polypeptide is a fusion polypeptide.

10. The composition of any one of claims 1-7, wherein said nucleic acid molecule is selected from hybridization probes, primers and anti-sense oligonucleotides.

11. The composition of any one of claims 1-10 which is a diagnostic composition.

12. The composition of any one of claims 1-10 which is a therapeutic composition.

13. The composition of any one of claims 1-12 for the manufacture of an agent for detecting and/or verifying, for the treatment, alleviation and/or prevention of an disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis and/or gallstones and others, in cells, cell masses, organs and/or subjects.

14. Use of a nucleic acid molecule of the SCAD gene family or a polypeptide encoded thereby or a fragment or a variant of said nucleic acid molecule or said polypeptide or an effector, e.g. an antibody, an aptamer or another receptor recognizing a nucleic acid molecule of the SCAD gene family or a polypeptide encoded thereby for controlling the function of a gene and/or a gene product which is influenced and/or modified by a SCAD homologous polypeptide.

15. Use of the nucleic acid molecule of the SCAD gene family or a polypeptide encoded thereby or a fragment or a variant of said nucleic acid molecule or said polypeptide or an efector, e.g. an antibody, an aptamer or another receptor recognizing a nucleic acid molecule of the SCAD gene family or a polypeptide encoded thereby for identifying substances capable of interacting with a SCAD homologous polypeptide.

16. A non-human transgenic animal exhibiting a modified expression of a SCAD homologous polypeptide.

17. The animal of claim 16, wherein the expression of the SCAD homologous polypeptide is increased and/or reduced.

18. A recombinant host cell exhibiting a modified expression of a SCAD homologous polypeptide.

19. The cell of claim 18 which is a human cell.

20. A method of identifying a (poly)peptide involved in the regulation of energy homeostasis and/or metabolism of triglycerides in a mammal comprising the steps of
(a) contacting a collection of (poly)peptides with a SCAD homologous polypeptide or a fragment thereof under conditions that allow binding of said (poly)peptides;
(b) removing (poly)peptides which do not bind and
(c) identifying (poly)peptides that bind to said SCAD homologous polypeptide.

21. A method of screening for an agent which modulates the interaction of a SCAD homologous polypeptide with a binding target/agent, comprising the steps of
(a) incubating a mixture comprising
(aa) a SCAD homologous polypeptide, or a fragment thereof;
(ab) a binding target/agent of said SCAD homologous polypeptide or fragment thereof; and
(ac) a candidate agent
under conditions whereby said SCAD polypeptide or fragment thereof specifically binds to said binding target/agent at a reference affinity;
(b) detecting the binding affinity of said SCAD polypeptide or fragment thereof to said binding target to determine an (candidate) agent-biased affinity; and
(c) determining a difference between (candidate) agent-biased affinity and the reference affinity.

22. A method of screening for an agent which modulates the activity of a SCAD homologous polypeptide, comprising the steps of
(a) incubating a mixture comprising
(aa) a SCAD homologous polypeptide or a fragment thereof; and
(ab) a candidate agent
under conditions whereby said SCAD polypeptide fragment thereof has a reference activity;
(b) detecting the activity of said SCAD polypeptide or fragment thereof to determine an (candidate) agent-biased activity; and
(c) determining a difference between (candidat) agent-biased activity and reference activity.

23. A method of producing a composition comprising the (poly)peptide identified by the method of claim 20 or the agent identified by the method of claim 21 or 22 with a pharmaceutically acceptable carrier, diluent and/or adjuvant.

24. The method of claim 23 wherein said composition is a pharmaceutical composition for preventing, alleviating or treating of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis and/or gallstones and other diseases and disorders.

25. Use of a (poly)peptide as identified by the method of claim 20 or of an agent as identified by the method of claim 0.21 or 22 for the preparation of a pharmaceutical composition for the treatment, alleviation and/or prevention of of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, and/or gallstones and other diseases and disorders.

26. Use of a nucleic acid molecule of the SCAD family or of a fragment thereof for the preparation of a non-human animal which over- or under-expresses the CG3842 homologous gene product.

27. Kit comprising at least one of
(a) a SCAD nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).
